(19) United States
(12) Patent Application Publication
(10) Pub. No.: US 2007/0142675 A1
(43) Pub. Date: Jun. 21, 2007

(54) Method for producing c1-c15 fragments of epothilones and the derivatives thereof

(76) Inventors: Ulrich Klar, Berlin (DE); Bernd Buchmann, Hohen Neuendorf (DE); Wolfgang Schwede, Glienicke (DE); Werner Skuballa, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/563,058

(22) PCT Filed: Jun. 19, 2004
(86) PCT No.: PCT/EP04/06685

§ 371 Date: Jun. 19, 2006

(30) Foreign Application Priority Data
Jul. 3, 2003 (DE) 103 31 004.5

(51) Int. Cl. C07C 45/45

(52) U.S. Cl. 568/312

(57) ABSTRACT

This invention describes a process for the production of C₁-C₁₅-fragments of epothilones and derivatives thereof, in which a C1-C6-fragment is linked with a C7-C12-fragment to a C1-C12-fragment, and the latter then is reacted with a C13-15-fragment to form the C1-C15 initial epothilone product that is to be produced. The thus obtained C1-C15 initial epothilone products can be reacted according to known methods to form the actual active ingredients. In addition, the invention relates to the corresponding C1-C12-fragments.

Höfle et al. describe the cytotoxic action of the natural substances epothilone A (R=hydrogen) and epothilone B (R=methyl) 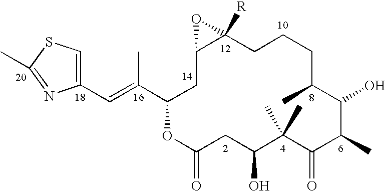
Epothilone A (R═H), Epothilone B (R═CH₃)
   in, e.g., Angew. Chem. [Applied Chem.] 1996, 108, 1671-1673. Because of the in-vitro selectivity for breast cell lines and intestinal cell lines and their significantly higher activity against P-glycoprotein-forming multiresistant tumor lines in comparison to taxol as well as their physical properties that are superior to those of taxol, e.g., a water solubility that is higher by a factor of 30, this novel structural class is especially advantageous for the development of a pharmaceutical agent for therapy of malignant tumors.

The object of this invention consists in making available new C1-C12-epothilone components in large amounts that can be used for the synthesis of a wide variety of epothilones and derivatives thereof, as they are described in, for example, WO 9907692, WO 0049020, WO 0001333 or DE 199210861.

This invention describes the novel production of the C₁-C₁₅-epothilone fragment of general formula I,

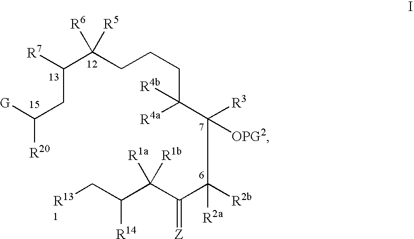

in which
R^{1a}, R^{1b} are the same or different and mean hydrogen, C₁-C₁₀-alkyl, aryl, C₇-C₂₀-aralkyl, or together mean a —(CH₂)ₘ group with m=2, 3, 4 or 5,
R^{2a}, R^{2b} are the same or different and mean hydrogen, C₁-C₁₀-alkyl, C₂-C₁₀-alkenyl, C₂-C₁₀-alkinyl, aryl, C₇-C₂₀-aralkyl or together mean a —(CH₂)ₙ group with n=2, 3, 4 or 5,
R³ means hydrogen, C₁-C₁₀-alkyl, aryl, or C₇-C₂₀-aralkyl,
R^{4a}, R^{4b} are the same or different and mean hydrogen, C₁-C₁₀-alkyl, aryl, C₇-C₂₀-aralkyl or together mean a —(CH₂)ₚ group with p=2, 3, 4 or 5,
R⁵ means hydrogen, C₁-C₁₀-alkyl, aryl, or C₇-C₂₀-aralkyl,
R⁶, R⁷ each mean a hydrogen atom, together an additional bond or together an oxygen atom,
G means a group X═CR⁸, a bicyclic or tricyclic aryl radical,
R⁸ means hydrogen, halogen, C₁-C₂₀-alkyl, aryl, or C₇-C₂₀-aralkyl, which can all be substituted,
X means an oxygen atom, two alkoxy groups OR²³, a C₂-C₁₀-alkylene-α,ω-dioxy group, which can be straight-chain or branched, H/OR⁹ or a grouping CR¹⁰R¹¹,
   whereby
   R²³ stands for a C₁-C₂₀-alkyl radical,
   R⁹ stands for hydrogen or a protective group PG^{X},
   R¹⁰, R¹¹ are the same or different and stand for hydrogen, a C₁-C₂₀-alkyl radical, aryl radical, or C₇-C₂₀-aralkyl radical, or R¹⁰ and R¹¹ together with the methylene carbon atom together stand for a 5- to 7-membered carbocyclic ring,
R¹³ means CH₂OR^{13a}, CH₂-Hal, CHO, CO₂R^{13b}, or COHal,
R¹⁴ means hydrogen, OR^{14a}, Hal, or OSO₂R^{14b},
R^{13a}, R^{14a} mean hydrogen, SO₂-alkyl, SO₂-aryl, SO₂-aralkyl or together a —(CH₂)ₒ group or together a CR^{15a}R^{15b} group,
R^{13b}, R^{14b} mean hydrogen, C₁-C₂₀-alkyl, aryl, or C₁-C₂₀-aralkyl,
R^{15a}, R^{15b} are the same or different and mean hydrogen, C₁-C₁₀-alkyl, aryl, C₇-C₂₀-aralkyl, or together a —(CH₂)_{q} group,
o means 2 to 4,
q means 3 to 6,
R²⁰ means OPG³, NHR²⁹, or N₃,
Z means an oxygen atom or H/OR¹²,
   whereby
   R¹² is hydrogen or a protective group PG^{Z}

including all stereoisomers as well as mixtures thereof, and

free hydroxyl groups in R¹³ and R¹⁴ can be etherified or esterified, free carbonyl groups in Z and R¹³ can be ketalized, converted into an enol ether or reduced, and free acid groups in R¹³ and R¹⁴ can be converted into their salts with bases.

As alkyl groups R^{1a}, R^{1b}, R^{2a}, R^{2b}, R³, R^{4a}, R^{4b}, R⁵, R⁸, R¹⁰, R¹¹, R^{13b}, R^{14b}, R^{15a}, R^{15b} and R²³, straight-chain or branched-chain alkyl groups with 1-10 carbon atoms can be considered, such as, for example, methyl, ethyl, propyl, isopropyl, butyl, isobutyl, tert.-butyl, pentyl, isopentyl, neopentyl, heptyl, hexyl, and decyl.

Alkyl groups R^{1a}, R^{1b}, R^{2a}, R^{2b}, R³, R^{4a}, R^{4b}, R⁵, R⁸, R¹⁰, R¹¹, R^{13b}, R^{14b}, R^{15a}, R^{15b} and R²³ can be perfluorinated or substituted by 1-5 halogen atoms, hydroxy groups, C₁-C₄-alkoxy groups, or C₆-C₁₂-aryl groups (which can be substituted by 1-3 halogen atoms).

As aryl radicals R^{1a}, R^{1b}, R^{2a}, R^{2b}, R³, R^{4a}, R^{4b}, R⁵, R⁸, R¹⁰, R¹¹, R^{13b}, R^{14b}, R^{15a} and R^{15b}, substituted and unsubstituted carbocyclic or heterocyclic radicals with one or more heteroatoms, such as, e.g., phenyl, naphthyl, furyl, thienyl, pyridyl, pyrazolyl, pyrimidinyl, oxazolyl, pyridazinyl, pyrazinyl, quinolyl, and thiazolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, C₁-C₂₀-acyloxy groups, are suitable.

The aralkyl groups in R^{1a}, R^{1b}, R^{2a}, R^{2b}, R³, R^{4a}, R^{4b}, R⁵, R⁸, R¹⁰, R¹¹, R^{13b}, R^{14b}, R^{15a} and R^{15b} can contain in the ring up to 14 C atoms, preferably 6 to 10 C atoms, and in the alkyl chain 1 to 8 atoms, preferably I to 4 atoms. As aralkyl radicals, for example, benzyl, phenylethyl, naphthylmethyl, naphthylethyl, furylmethyl, thienylethyl, and pyridylpropyl can be considered. The rings can be substituted in one or more places by halogen, OH, O-alkyl, CO₂H, CO₂-alkyl, —NO₂, —N₃, —CN, C₁-C₂₀-alkyl, C₁-C₂₀-acyl, and C₁-C₂₀-acyloxy groups.

As alkenyl groups R^{2a} and R^{2b}, straight-chain or branched-chain alkyl groups with 1-10 carbon atoms can be considered, in which at least one C—C bond is replaced by a C═C bond, such as, for example, propenyl, butenyl, isobutenyl, pentenyl, isopentenyl, neopentenyl, heptenyl, heptadienyl, decenyl, or decatrienyl.

As alkinyl groups R^{2a} and R^{2b}, straight-chain or branched-chain alkyl groups with 1-10 carbon atoms can be considered, in which at least one C—C bond is replaced by a C≡C bond, such as, for example, propinyl, butinyl, pentinyl, isopentinyl, heptinyl, heptadiinyl, decinyl, and decatriinyl.

Preferred are those compounds I in which
R^{1a}, R^{1b} are the same and mean C₁-C₆-alkyl, or together mean a —(CH₂)ₘ group with m=2, 3 or 4,
R^{2a}, R^{2b} are different and mean hydrogen, C₁-C₆-alkyl, C₂-C₁₀-alkenyl, C₂-C₁₀-alkinyl or C₇-C₂₀-aralkyl,
R⁵ means hydrogen or C₁-C₆-alkyl,
R⁸ means hydrogen, halogen, or C₁-C₆-alkyl,
R^{15a}, R^{15b} are the same or different and mean hydrogen, C₁-C₆-alkyl, aryl, C₇-C₂₀-aralkyl, or together a —(CH₂)_{q} group,
q means 3 to 6.
Especially preferred are those compounds I in which
R^{1a}, R^{1b} are the same and mean C₁-C₃-alkyl, or together mean a —(CH₂)ₘ group with m=2, 3 or 4,
R^{2a} a means hydrogen,
R^{2b} means C₁-C₅-alkyl, C₂-C₆-alkenyl, or C₂-C₆-alkinyl,
R⁵ means hydrogen or C₁-C₃-alkyl,
R⁶, R⁷ together mean an additional bond,
G means a group X═CR⁸, or a bicyclic aryl radical,
R⁸ means hydrogen, fluorine, chlorine, or C₁-C₃-alkyl,
X means oxygen or a group CR¹⁰R¹¹,
R¹⁰ means hydrogen,
R¹¹ means aryl,
R¹³ means CH₂OR^{13a}, or CO₂R^{13b},
R¹⁴ means OR^{14a},
R^{13a}, R^{14a} together mean a CR^{15a}R^{15b} group,
R^{13b} means hydrogen or C₁-C₆-alkyl,
R^{15a}, R^{15b} are the same and mean C₁-C₃-alkyl, or together mean a —(CH₂)_{q} group, or
R^{15a}, R^{15b} are different and mean hydrogen or aryl,
q means 4 or 5,
Z means oxygen.

The production of new epothilone derivatives is based on the linkage of three partial fragments A, B and C. The interfaces lie as indicated in general formula I′.

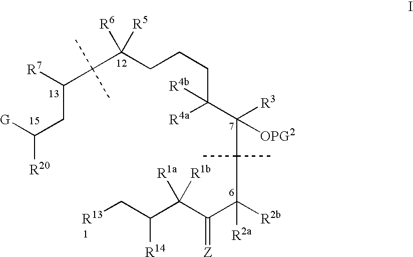

A means a C1-C6 fragment (epothilone numbering system) of general formula A-1

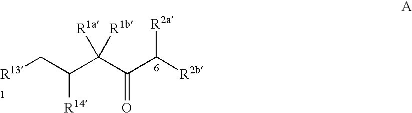

in which

R^{1a′}, R^{1b′}, R^{2a′}, R^{2b′}, R^{13′} and R^{14′} have the meanings that are already mentioned for R^{1a}, R^{1b}, R^{2a}, R^{2b}, R¹³ and R¹⁴, including all stereoisomers as well as mixtures thereof, and

free hydroxyl groups in R¹³ and R¹⁴ can be etherified or esterified, free carbonyl groups in A and R¹³ can be ketalized, converted into an enol ether or reduced, and free acid groups in A can be converted into their salts with bases.

B stands for a C7-C12 fragment (epothilone numbering system) of general formula

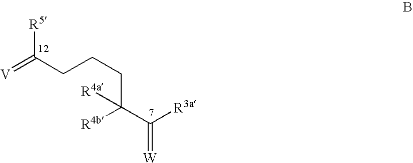

in which
R^{3a′}, R^{4a′}, R^{4b′} and R^{5′} have the meanings that are already mentioned for R^{3a}, R⁴ and R⁵, and
V means an oxygen atom, two alkoxy groups OR¹⁷, a C₂-C₁₀-alkylene-α,ω-dioxy group, which can be straight-chain or branched, or H/OR¹⁶,
W means an oxygen atom, two alkoxy groups OR¹⁹, a C₂-C₁₀-alkylene-α,ω-dioxy group, which can be straight-chain or branched, or H/OR¹⁸,
R¹⁶, R¹⁸, independently of one another, mean hydrogen or a protective group PG¹,
R¹⁷, R¹⁹, independently of one another, mean C₁-C₂₀-alkyl.

C stands for a C13-C15-fragment (epothilone numbering system) of general formula

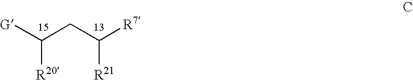

in which
G′ has the meaning already mentioned in general formula I for G, and
R^{7′} means a hydrogen atom,
R^{20′} means halogen, N₃, NHR²⁹, a hydroxy group, a protected hydroxy group O-PG³, a protected amino group NR²⁹PG³, a C₁-C₁₀-alkylsulfonyloxy group, which optionally can be perfluorinated, a benzoyloxy group that is optionally substituted by C₁-C₄-alkyl, nitro, chlorine or bromine, an NR²⁹SO₂CH₃ group, an NR²⁹C(═O)CH₃ group, or a CH₂—C(═O)—CH₃ group,
R²¹ means a hydroxy group, halogen, a protected hydroxy group OPG³, a phosphonium halide radical PPh₃⁺Hal⁻ (Ph=phenyl; Hal=F, Cl, Br, I), a phosphonate radical P(O)(OQ)₂ (Q=C₁-C₁₀-alkyl or phenyl) or a phosphine oxide radical P(O)Ph₂ (Ph=Phenyl),
R²⁹ means hydrogen or C₁-C₆-alkyl.

As alkyl groups R^{1a}, R^{1b}, R^{2a}, R^{2b}, R³, R^{4a}, R^{4b}, R⁵, R⁸, R¹⁰, R¹¹, R^{13b}, R^{14b}, R^{15a}, R^{15b}, R¹⁷, R¹⁹, R²³ and R²⁹, straight-chain or branched-chain alkyl groups with 1-20 carbon atoms can be considered, such as, for example, methyl, ethyl, propyl, isopropyl, butyl, isobutyl, tert.-butyl, pentyl, isopentyl, neopentyl, heptyl, hexyl, and decyl.

Alkyl groups R^{1a}, R^{1b}, R^{2a}, R^{2b}, R³, R^{4a}, R^{4b}, R⁵, R⁸, R¹⁰, R¹¹, R^{13b}, R^{15a}, R^{15b}, R¹⁷, R¹⁹, R²³ and R²⁹ can be perfluorinated or substituted by 1-5 halogen atoms, hydroxy groups, C₁-C₄-alkoxy groups, or C₆-C₁₂-aryl groups (which can be substituted by 1-3 halogen atoms).

As aryl radicals R^{1a}, R^{1b}, R^{2a}, R^{2b}, R³, R^{4a}, R^{4b}, R⁵, R⁸, R¹⁰, R¹¹, R^{13b}, R^{14b}, R^{15a} and R^{15b}, substituted and unsubstituted carbocyclic or heterocyclic radicals with one or more heteroatoms, such as, e.g., phenyl, naphthyl, furyl, thienyl, pyridyl, pyrazolyl, pyrimidinyl, oxazolyl, pyridazinyl, pyrazinyl, quinolyl, thiazolyl, benzothiazolyl, benzoxazolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, or C₁-C₂₀-acyloxy groups, are suitable.

As bicyclic and tricyclic aryl radicals G, substituted and unsubstituted carbocyclic or heterocyclic radicals with one or more heteroatoms, such as, e.g., naphthyl, anthryl, benzothiazolyl, benzoxazolyl, benzimidazolyl, quinolyl, isoquinolyl, benzoxazinyl, benzofuran, indolyl, indazolyl, quinoxalinyl, tetrahydroisoquinolinyl, tetrahydroquinolinyl, thienopyridinyl, pyridopyridinyl, benzopyrazolyl, benzotriazolyl, dihydroindolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, or C₁-C₂₀-acyloxy groups, are suitable.

The aralkyl groups in R^{1a}, R^{1b}, R^{2a}, R^{2b}, R³, R^{4a}, R^{4b}, R⁵, R⁸, R¹⁰, R¹¹, R^{13b}, R^{14b}, R^{15a} and R^{15b} can contain up to 14 C atoms, preferably 6 to 10 C atoms, in the ring, and 1 to 8 atoms, preferably 1 to 4 atoms, in the alkyl chain. As aralkyl radicals, for example, benzyl, phenylethyl, naphthylmethyl, naphthylethyl, furylmethyl, thienylethyl, and pyridylpropyl are considered. The rings can be substituted in one or more places by halogen, OH, O-alkyl, CO₂H, CO₂-alkyl, —NO₂, —N₃, —CN, C₁-C₂₀-alkyl, C₁-C₂₀-acyl, or C₁-C₂₀-acyloxy groups.

As representatives of protective groups PG, alkyl- and/or aryl-substituted silyl, C₁-C₂₀-alkyl, C₄-C₇-cycloalkyl, which in addition can contain an oxygen atom in the ring, aryl, C₇-C₂₀-aralkyl, C₁-C₂₀-acyl as well as aryl can be mentioned.

As alkyl-, silyl- and acyl radicals for the protective groups PG, the radicals that are known to one skilled in the art are considered. Alkyl or silyl radicals that can easily be cleaved from the corresponding alkyl and silyl ethers, such as, for example, methoxymethyl, methoxyethyl, ethoxyethyl, tetrahydropyranyl, tetrahydrofuranyl, trimethylsilyl, triethylsilyl, tert.-butyldimethylsilyl, tert.-butyldiphenylsilyl, tribenzylsilyl, triisopropylsilyl, benzyl, para-nitrobenzyl, and para-methoxybenzyl radicals as well as alkylsulfonyl and arylsulfonyl radicals are preferred. As acyl radicals, e.g., formyl, acetyl, propionyl, isopropionyl, pivalyl, butyryl or benzoyl, which can be substituted with amino groups and/or hydroxy groups, are suitable.

As amino protective groups, the radicals that are known to one skilled in the art are suitable. For example, the alloc, boc, Z, benzyl, f-moc, troc, stabase or benzostabase group can be mentioned.

Acyl groups PG can contain 1 to 20 carbon atoms, whereby formyl, acetyl, propionyl, isopropionyl and pivalyl groups are preferred.

Index m in the alkylene group that is formed from R^{1a} and R^{1b} preferably stands for 1, 2, 3 or 4.

The C₂-C₁₀-alkylene-α,ω-dioxy group that is possible for V, W and X is preferably an ethyleneketal or neopentylketal group.

Production of Partial Fragments A:

The partial fragments (synthesis components) of general formula A can be produced, for example, as described in WO 99/07692 or DE 101 64 592.9.

Production of Partial Fragments B:

The partial fragments (synthesis components) of general formula B can be produced, for example, as described in WO 99/07692.

Production of Partial Fragments C:

The partial fragments (synthesis components) of general formula C can be produced, for example, as described in DE 197 51 200.3, DE 199 07 480.1, WO 99/07692 and WO 00/01333.
Partial Fragments of General Formula AB 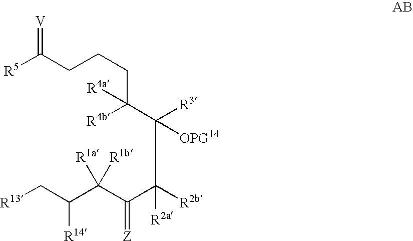
in which R^{1a′}, R^{1b′}, R^{2a′}, R^{2b′}, R^{3′}, R^{4a′}, R^{4b′}, R⁵, R^{13′}, R^{14′}, V and Z have the already mentioned meanings, and PG¹⁴ represents a hydrogen atom or a protective group PG, are obtained from the previously mentioned fragments A and B according to the process that is shown in Diagram 1. 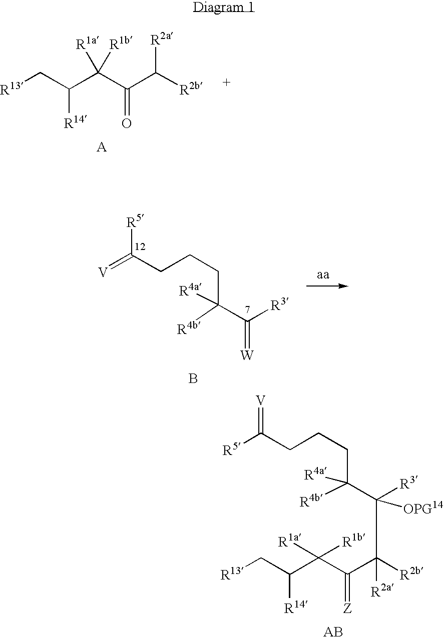
Step aa (A+B [symbol] AB):

Compound B, in which W has the meaning of an oxygen atom, and an optionally present additional carbonyl group in V and/or an optionally present additional hydroxy group in V (H/OR¹⁶) are protected, is alkylated with the enolate of a carbonyl compound of general formula A, optionally in the presence of metal halides. The enolate is produced by the action of strong bases, such as, e.g., lithium diisopropyl amide, or lithium hexamethyldisilazane, at low temperatures.
Partial Fragments of General Formula ABC (AB+C) 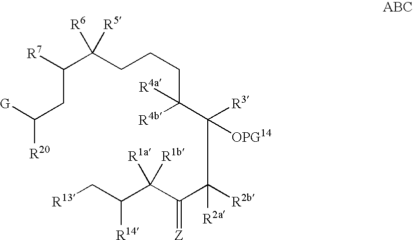
in which R^{1a′}, R^{1b′}, R^{2a′}, R^{2b′}, R^{3′}, R^{4a′}, R^{4b′}, R^{5′}, R⁶, R⁷, R¹³, R¹⁴, G and Z have the already mentioned meanings, and PG¹⁴ represents a hydrogen atom or a protective group PG, are obtained from the previously described fragments AB and C according to the process that is shown in Diagram 2. 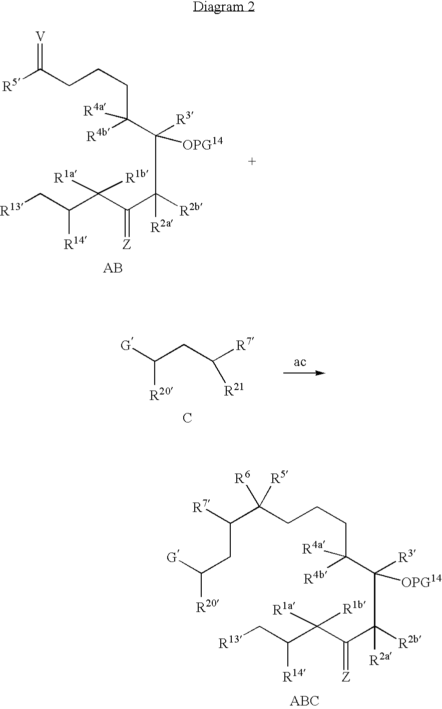
Step ac (AB+C [symbol] ABC):

Compound C, in which R²¹ has the meaning of a phosphonium halide radical PPh₃⁺Hal⁻, preferably a PPh₃⁺I⁻ radical or a phosphonate radical or a phosphine oxide radical, and optionally present additional carbonyl groups are optionally protected, is deprotonated by a suitable base, such as, e.g., n-butyllithium, lithium diisopropylamide, potassium-tert.butanolate, sodium- or lithium-hexamethyldisilazide and reacted with a compound AB, in which V has the meaning of an oxygen atom.

The thus obtained fragment ABC, which contains ring carbon atoms C1 to C15 of the later 16-membered macrocyclic compound, is converted into the desired target compounds, as they are mentioned in the Patent Applications of the same name, for example according to the processes that are described in WO 99/07692, WO 99/049154 or WO 00/01333.

The invention therefore also relates to a process for the production of the epothilone derivatives of general formula II 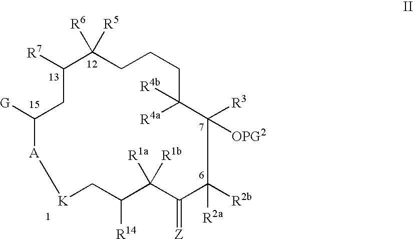
in which A-K means a group —O—C(═O), —OCH₂—, —CH₂C(═O)—, —NR²⁹—C(═O)—, —NR²⁹—SO₂—, and substituents R^{1a}, R^{1b}, R^{2a}, R^{2b}, R³, R^{4a}, R^{4b}, R⁵, R⁶, R⁷, G, OPG² and Z have the meanings that are indicated in general formula I,
in which compounds of general formula I, obtained according to the process according to the invention, are cyclized to compounds of general formula II.

By the process that is described here (A+B [symbol] AB+C [symbol] ABC), it has recently become possible to introduce fragment C that is more synthetically expensive to produce. As a result, the amounts of required fragment C are reduced in comparison to the linkage sequence C+B [symbol] BC+A [symbol] ABC, which is advantageous both economically and ecologically for the production of fragment ABC.

### EXAMPLE 1

### (3S,6R,7S,8S,12Z,15S)-4,4,8,12-Tetramethyl-5-oxo-6-allyl-3,7,15-tris-(tert.-butyldimethylsilyloxy)-15-(2-methylbenzothiazol-5-yl)-pentadec-12-enoic acid

### EXAMPLE 1a

### (4S,4′R,5′S,6′S,10′RS)-4-(2,6-Dimethyl-3-oxo-4-allyl-5-hydroxy-10-(tert.-butyldiphenylsilyloxy)-undecan-2-yl)-2,2-dimethyl-1,3-dioxane (A) and (4S,4′R,5′S,6′S,10′RS)-4-(2,6-Dimethyl-3-oxo-4-allyl-5-hydroxy-10-(tert.-butyldiphenylsilyloxy)-undecan-2-yl)-2,2-dimethyl-1,3-dioxane (B)

The solution of 13.8 ml of diisopropylamine in 350 ml of anhydrous tetrahydrofuran is mixed at −30° C. under an atmosphere of dry argon with 39.5 ml of a 2.5 molar solution of n-buthyllithium in n-hexane, stirred for 30 minutes and cooled to −70° C. Within 20 minutes, the solution of 22.6 g (94 mmol) of (4S)-4-(2-methyl-3-oxo-hept-6-en-2-yl)-2,2-dimethyl-1,3-dioxane, which was produced analogously to the process described in WO 99/07692, WO 99049154, and WO 00/01333, in 350 ml of tetrahydrofuran, is added in drops, and it is allowed to heat within 2 hours to −30° C. Then, it is cooled again to −70° C., mixed with the solution of 12.8 g of anhydrous zinc chloride in 130 ml of tetrahydrofuran, and after 15 minutes, the solution of 16.8 g (43.9 mmol) of m(2S,6RS)-2-methyl-6-(tert.-butyl-diphenylsilyloxy)-heptanal, which was produced analogously to the process described in WO 99/07692, WO 99049154, and WO 00/01333, in 400 ml of tetrahydrofuran is added in drops. It is stirred for another 2.5 hours at −70° C., poured into a saturated ammonium chloride solution and extracted several times with ethyl acetate. The combined organic extracts are washed with water and saturated sodium chloride solution, dried on sodium sulfate, and the residue obtained after filtration and removal of the solvent is purified by chromatography on fine silica gel with a mobile solvent mixture that consists of n-hexane and ethyl acetate. 19.8 g (31.8 mmol, 72%) of title compound A, 2.28 g (3.7 mmol, 8%) of title compound B as well as 12.5 g of (4S)-4-(2-methyl-3-oxo-hept-6-en-2-yl)-2,2-dimethyl-1,3-dioxane are isolated in each case as a colorless oil.

¹H-NMR (CDCl₃) of A: δ=0.80 (3H), 0.96-1.06 (16H), 1.10-1.74 (4H), 1.23 (3H), 1.30 (3H), 1.37 (3H), 1.57 (3H), 2.21 (1H), 2.43 (1H), 2.88 (1H), 3.32 (1H), 3.44 (1H), 3.86 (2H), 3.97 (1H), 4.12 (2H), 4.97 (1H), 5.03 (1H), 5.70 (1H), 7.31-7.44 (6H), 7.64-7.70 (4H) ppm.

¹H-NMR (CDCl₃) of B: δ=0.89-1.55 (36H), 1.66 (1H), 2.30 (1H), 2.40 (1H), 2.66 (1H), 3.28 (1H), 3.51 (1H), 3.78-4.17 (3H), 4.97 (1H), 5.03 (1H), 5.70 (1H), 7.32-7.46 (6H), 7.64-7.71 (4H) ppm.

### EXAMPLE 1b

### (4S,4′R,5′S,6′S,10′RS)-4-(2,6-Dimethyl-3-oxo-4-allyl-5-(2H-tetrahydropyran-2-yloxy)-10-(tert.-butyldiphenylsilyloxy)-undecan-2-yl)-2,2-dimethyl-1,3-dioxane

The solution of 19.7 g (31.7 mmol) of compound A, produced according to Example 1a, in 500 ml of anhydrous dichloromethane is mixed with 40 ml of 3,4-dihydro-(2H)-pyran and 1.6 g of p-toluenesulfonic acid-pyridinium salt, and it is stirred for 2 days at 23° C. It is poured into a saturated sodium bicarbonate solution, extracted with dichloromethane, and the combined organic extracts are dried on sodium sulfate. After filtration and removal of the solvent, the residue is purified by chromatography on fine silica gel with a mobile solvent mixture that consists of n-hexane and ethyl acetate. 22.0 g (31.1 mmol, 98%) of the title compound is isolated as a colorless oil.

¹H-NMR (CDCl₃): δ=0.87-1.91 (42H), 2.14-2.41 (1H), 2.57 (1H), 3.14-4.28 (8H), 4.40-4.53 (1H), 4.90-5.05 (2H), 5.58-5.89 (1H), 7.31-7.45 (6H), 7.64-7.70 (4H) ppm.

### EXAMPLE 1c

### (4S,4′R,5′S,6′S,10′RS)-4-(2,6-Dimethyl-3-oxo-4-allyl-5-(2H-tetrahydropyran-2-yloxy-10-hydroxy-undecan-2-yl)-2,2-dimethyl-1,3-dioxane

The solution of 22.0 g (31.1 mmol) of the compound, produced according to Example 1b, in 400 ml of tetrahydrofuran is mixed with 62.3 ml of a 1 molar solution of tetrabutylammonium fluoride in tetrahydrofuran, and it is stirred for 12 hours at 80° C. It is poured into a saturated sodium chloride solution, diluted with water and extracted several times with ethyl acetate. The combined organic phases are dried on sodium sulfate, and the residue that is obtained after filtration and removal of the solvent is purified by chromatography on fine silica gel with a mobile solvent mixture that consists of n-hexane and ethyl acetate. 13.6 g (29.0 mmol, 93%) of the title compound is isolated as a colorless oil.

¹H-NMR (CDCl₃): δ=0.91-1.81 (34H), 2.14-2.42 (1H), 2.58 (1H), 3.15-3.46 (2H), 3.63-4.29 (6H), 4.38-4.57 (1H), 4.89-5.06 (2H), 5.57-5.88 (1H) ppm.

### EXAMPLE 1d

### (4S,4′R,5′S,6′S)-4-(2,6-Dimethyl-3,10-dioxo-4-allyl-5-(2H-tetrahydropyran-2-yloxy)-undecan-2-yl)-2,2-dimethyl-1,3-dioxane

The solution of 13.6 g (29.0 mmol) of the compound, produced according to Example 1c, in 480 ml of anhydrous dichloromethane is mixed with about 1 g of molecular sieve (4A), 6.06 g of N-methylmorpholine-N-oxide, and 400 mg of tetrapropylammonium perruthenate, and it is stirred for 18 hours at 23° C. It is concentrated by evaporation, and the residue is purified by chromatography on fine silica gel with a mobile solvent mixture that consists of n-hexane and ethyl acetate. 11.6 g (24.9 mmol, 86%) of the title compound is isolated as a colorless oil.

¹H-NMR (CDCl₃): δ=0.91-1.83 (29H), 2.13 (3H), 2.05-2.63 (4H), 3.15-4.02 (5H), 4.22 (1H), 4.40+4.51 (1H), 4.90-5.05 (2H), 5.57-5.88 (1H) ppm.

### EXAMPLE 1e

### (4S,4′R,5′S,6′S,10′E/Z,13′S)-4-(2,6,10-Trimethyl-3-oxo-4-allyl-5-(2H-tetrahydropyran-2-yloxy)-13-(2-methylbenzothiazol-5-yl)-13-(tert.-butyldimethylsilyloxy)-tridec-10-en-2-yl)-2,2-dimethyl-1,3-dioxane

The solution of 6.72 g of [(3S)-3-(2-methylbenzothiazol-5-yl)-3-(tert.-butyldimethylsilyloxy)-propyl]-triphenylphosphonium iodide, which was produced analogously to the process described in WO 99/07692, WO 99049154, and WO 00/01333, in 45 ml of anhydrous tetrahydrofuran is mixed at 0° C. with 9.5 ml of a 1 molar solution of sodium hexamethyldisilazane in tetrahydrofuran and then with the solution of 3.0 g (6.43 mmol) of the compound, produced according to Example 1d, in 45 ml of tetrahydrofuran. It is allowed to react for 5 hours at 23° C., poured into a saturated ammonium chloride solution and extracted several times with ethyl acetate. The combined organic extracts are washed with water and saturated ammonium chloride solution, dried on sodium sulfate, and the residue that is obtained after filtration and removal of the solvent is purified by chromatography on fine silica gel with a mobile solvent mixture that consists of n-hexane and ethyl acetate. 4.25 g (5.52 mmol, 86%) of the title compound is isolated as a colorless oil.

¹H-NMR (CDCl₃): δ=−0.12 (3H), 0.03 (3H), 0.88 (9H), 0.82-2.65 (37H), 2.83 (3H), 3.14-4.29 (7H), 4.40-4.56 (1H), 4.73 (1H), 4.88-5.04 (2H), 5.15 (1H), 5.58-5.89 (1H), 7.32 (1H), 7.72 (1H), 7.86 (1H) ppm.

### EXAMPLE 1f

### (3S,6R,7S,8S,12E/Z,15S)-4,4,8,12-Tetramethyl-5-oxo-6-allyl-15-(tert.-butyldimethylsilyloxy)-15-(2-methylbenzothiazol-5-yl)-pentadec-12-ene-1,3,7-triol

The solution of 10.3 g (13.4 mmol) of the compound, produced according to Example 1e, in 270 ml of ethanol is mixed with 5.33 g of p-toluenesulfonic acid-monohydrate, and it is stirred for 5 hours at 23° C. It is concentrated by evaporation, the residue is taken up in dichloromethane, washed with saturated sodium bicarbonate solution, and dried on sodium sulfate. The crude product that is obtained after filtration and removal of the solvent is further reacted without purification.

### Example 1g

### (3S,6R,7S,8S,12E/Z,15S)-4,4,8,12-Tetramethyl-6-allyl-1,3,7,15-tetrakis-(tert.-butyldimethylsilyloxy)-15-(2-methylbenzothiazol-5-yl)-pentadec-12-en-5-one

The solution of the crude product, produced according to Example 1f (maximum 13.4 mmol), in 340 ml of anhydrous dichloromethane is mixed at 0° C. with 20.8 ml of 2,6-lutidine, 20.2 ml of trifluoromethanesulfonic acid-(tert.-butyldimethylsilylester), and it is stirred for 4 hours at 0° C. to 23° C. It is washed with a I molar hydrochloric acid, then with saturated sodium bicarbonate solution, and it is dried on sodium sulfate. After filtration and removal of the solvent, the residue is purified by chromatography on fine silica gel with a mobile solvent mixture that consists of n-hexane and ethyl acetate. 12.0 g (12.1 mmol, 91%) of the title compound is isolated as a colorless oil.

¹H-NMR (CDCl₃): δ=−0.13-0.08 (24H), 0.88 (36H), 0.89-1.67 (19H), 1.91 (2H), 2.19-2.52 (4H), 2.83 (3H), 3.13 (1H), 3.49-3.71 (2H), 3.75 (1H), 3.85 (1H), 4.74 (1H), 4.92 (1H), 4.98 (1H), 5.15 (1H), 5.75 (1H), 7.32 (1H), 7.73 (1H), 7.86 (1H) ppm.

### EXAMPLE 1h

### (3S,6R,7S,8S,12E/Z,15S)-4,4,8,12-Tetramethyl-6-allyl-1-hydroxy-3,7,15-tris-(tert.-butyldimethylsilyloxy)-15-(2-methylbenzothiazol-5-yl)-pentadec-12-en-5-one

The solution of 12.0 g (12.1 mmol) of the compound, produced according to Example 1 g, in a mixture that consists of 205 ml of dichloromethane and 100 ml of methanol is mixed at 0° C. with 2.68 g of rac.-camphor-10-sulfonic acid, and it is stirred for 3 hours at 0° C. It is poured into a saturated sodium bicarbonate solution, extracted with dichloromethane, and the combined organic extracts are dried on sodium sulfate. After filtration and removal of the solvent, the residue is purified by chromatography on fine silica gel with a mobile solvent mixture that consists of n-hexane and ethyl acetate. 9.07 g (10.4 mmol, 86%) of the title compound is isolated as a colorless oil.

¹H-NMR (CDCl3): δ=−0.13 (3H), 0.01-0.11 (15H), 0.80-0.95 (30H), 0.99-1.68 (13H), 1.48+1.65 (3H), 1.86-2.00 (3H), 2.20-2.52 (4H), 2.83 (3H), 3.12 (1H), 3.65 (2H), 3.77 (1H), 4.03 (1H), 4.74 (1H), 4.91-5.03 (2H), 5.16 (1H), 5.75 (1H), 7.32 (1H), 7.73 (1H), 7.86 (1H) ppm.

### EXAMPLE 1i

### (3S,6R,7S,8S, 12E/Z, 15S)-4,4,8,12-Tetramethyl-5-oxo-6-allyl-3,7,15-tris-(tert.-butyldimethylsilyloxy)-15-(2-methylbenzothiazol-5-yl)-pentadec-12-enal

The solution of 1.81 ml of oxalyl chloride in 95 ml of anhydrous dichloromethane is mixed at −70° C. with the solution of 2.94 ml of dimethyl sulfoxide in 10 ml of dichloromethane and after 10 minutes with the solution of 9.07 g (10.4 mmol) of the compound, produced according to Example 1h, in 95 ml of dichloromethane. It is stirred for 30 minutes at −70° C., mixed with 9.2 ml of triethylamine and stirred for another 30 minutes at −20° C. It is poured into a saturated sodium bicarbonate solution, extracted with dichloromethane, and the combined organic extracts are dried on sodium sulfate. After filtration and removal of the solvent, the residue is purified by chromatography on fine silica gel with a mobile solvent mixture that consists of n-hexane and ethyl acetate. 8.83 g (10.1 mmol, 97%) of the title compound is isolated as a pale yellow oil.

### EXAMPLE 1k

### (3S,6R,7S,8S,12Z,15S)-4,4,8,12-Tetramethyl-5-oxo-6-allyl-3,7,15-tris-(tert.-butyldimethylsilyloxy)-15-(2-methylbenzothiazol-5-yl)-pentadec-12-enoic acid (A) and (3S,6R,7S,8S,12E,15S)-4,4,8,12-Tetramethyl-5-oxo-6-allyl-3,7,15-tris-(tert.-butyldimethylsilyloxy)-15-(2-methylbenzothiazol-5-yl)-pentadec-12-enoic acid (B)

The solution of 8.83 g (10.1 mmol) of the compound, produced according to Example 1i, in a mixture that consists of 310 ml of tert-butanol, 235 ml of tetrahydrofuran and 78 ml of water is mixed at 0° C. with 4.41 g of sodium dihydrogen phosphate-monohydrate, 70 ml of 2-methyl-2-butene, and 7.5 g of sodium chlorite, and it is stirred for 1 hour at 0° C. It is poured into a saturated sodium thiosulfate solution, extracted with ethyl acetate, and the combined organic extracts are dried on sodium sulfate. After filtration and removal of the solvent, the residue is purified by chromatography on fine silica gel with a mobile solvent mixture that consists of n-hexane and ethyl acetate. 3.86 g (4.34 mmol, 43%) of title compound A as well as 3.96 g (4.46 mmol, 44%) of title compound B are isolated in each case as a colorless oil.

¹H-NMR (CDCl₃) of A: δ=−0.12 (3H), −0.03 (3H), 0.00 (3H), 0.07 (6H), 0.15 (3H), 0.85-0.90 (27H), 0.96 (3H), 1.03-1.95 (4H), 1.06 (3H), 1.18 (3H), 1.73 (3H), 1.74-1.95 (3H), 2.22-2.60 (7H), 2.83 (3H), 3.20 (1H), 3.66 (1H), 4.46 (1H), 4.79 (1H), 4.93 (1H), 4.99 (1H), 5.29 (1H), 5.71 (1H), 7.47 (1H), 7.75 (1H), 8.28 (1H) ppm.

¹H-NMR (CDCl₃) of B: δ=−0.10 (3H), 0.01 (6H), 0.06 (3H), 0.12 (3H), 0.16 (3H), 0.89 (30H), 1.00-1.48 (5H), 1.11 (3H), 1.21 (3H), 1.34 (3H), 1.76-2.03 (2H), 2.25-2.64 (6H), 2.84 (3H), 3.24 (1H), 3.80 (1H), 4.35 (1H), 4.67 (1H), 4.93-5.04 (2H), 5.12 (1H), 5.76 (1H), 7.46 (1H), 7.75 (1H), 7.82 (1H) ppm.

### EXAMPLE 2

### (3S,6R,7S,8S,12Z,15S)-4,4,8,12-Tetramethyl-5-oxo-6-allyl-3,7,15-tris-(tert.-butyldimethylsilyloxy)-15-(2-methylbenzothiazol-5-yl)-pentadec-12-enoic acid

### EXAMPLE 2a

### (2S)-2-Methyl-6-oxo-heptanol

The solution of 20 g (87.6 mmol) of (2S)-2-methyl-6-oxo-heptane-1-(tetrahydropyran-2-yloxy), which was produced analogously to the process described in DE 197 51 200.3, in 400 ml of anhydrous ethanol is mixed with 8.33 g of p-toluenesulfonic acid-monohydrate, and it is stirred for 2.5 hours at 23° C. It is concentrated by evaporation, the residue is taken up in dichloromethane, washed with saturated sodium bicarbonate solution and dried on sodium sulfate. The residue that is obtained after filtration and removal of the solvent is purified by chromatography on fine silica gel with a mixture that consists of n-hexane and ethyl acetate. 10.6 g (73.5 mmol, 83.9%) of the title compound is isolated as a colorless oil.

¹H-NMR (CDCl₃): δ=0.93 (3H), 1.13 (1H), 1.41 (1H), 1.48-1.76 (4H), 2.14 (3H), 2.45 (2H), 3.48 (2H) ppm.

### EXAMPLE 2b

### 2-Methyl-2-((2S)-2-methyl-1-hydroxy-pent-5-yl)-1,3-dioxolane

The solution of 10.6 g (73.5 mmol) of the compound, produced according to Example 2a, in 550 ml of anhydrous toluene is mixed with 50 ml of ethylene glycol and catalytic amounts of p-toluenesulfonic acid-monohydrate, and it is refluxed for 3 hours in a water separator. After cooling, it is diluted with ethyl acetate, washed with saturated sodium bicarbonate solution and water and dried on sodium sulfate. The residue that is obtained after filtration and removal of the solvent is purified by chromatography on fine silica gel with a mixture that consists of n-hexane and ethyl acetate. 11.6 g (61.6 mmol, 83.8%) of the title compound is isolated as a colorless oil.

¹H-NMR (CDCl₃): δ=0.92 (3H), 1.12 (1H), 1.31 (3H), 1.32-1.69 (5H), 3.42 (2H), 3.50 (2H), 3.93 (4H) ppm.

### EXAMPLE 2c

### 2-Methyl-2-((2S)-2-methyl-1-oxo-pent-5-yl)-1,3-dioxolane

The solution of 3.63 ml of oxalyl chloride in 200 ml of dichloromethane is cooled under an atmosphere of dry argon to −70° C., mixed with the solution of 5.92 ml of dimethyl sulfoxide in 20 ml of dichloromethane, and after 10 minutes, the solution of 5.0 g (26.6 mmol) of the compound, produced according to Example 2b, in 200 ml of dichloromethane, is added in drops. After 45 minutes, it is mixed with 18.3 ml of triethylamine and allowed to heat within one hour to 0° C. It is washed with water and saturated sodium chloride solution and dried on sodium sulfate. The residue that is obtained after filtration and removal of the solvent is taken up in a little diethyl ether, filtered on Celite and concentrated by evaporation. 5.1 g (maximum 26.2 mmol) of the title compound is isolated as a pale yellow oil that is further reacted without purification.

¹H-NMR (CDCl₃) of a purified analytical sample: δ=1.09 (3H), 1.30 (3H), 1.32-1.77 (6H), 2.34 (1H), 3.92 (4H), 9.61 (1H) ppm.

### EXAMPLE 2d

### (4S,4′R,5′S,6′S,10′RS)-4-[2,6-Dimethyl-3-oxo-4-allyl-5-hydroxy-9-(2-methyl-1,3-dioxolan-2-yl)-nonan-2-yl]-2,2-dimethyl-1,3-dioxane (A) and (4S,4′S,5′R,6′S,10′RS)-4-[2,6-Dimethyl-3-oxo-4-allyl-5-hydroxy-9-(2-methyl-1,3-dioxolan-2-yl)-nonan-2-yl]-2,2-dimethyl-1,3-dioxane (B)

The solution of 9.71 ml of diisopropylamine in 200 ml of anhydrous tetrahydrofuran was mixed at −30° C. under an atmosphere of dry argon with 27.7 ml of a 2.5 molar solution of n-buthyllithium in n-hexane, stirred for 30 minutes and cooled to −70° C. Within 20 minutes, the solution of 15.96 g (66.4 mmol) of (4S)-4-(2-methyl-3-oxo-hept-6-en-2-yl)-2,2-dimethyl-1,3-dioxane, which was produced analogously to the process described in WO 99/07692, WO 99049154, and WO 00/01333, in 200 ml of tetrahydrofuran is added in drops and allowed to heat within 2 hours to −30° C. Then, it is cooled again to −70° C., mixed with the solution of 9.04 g of anhydrous zinc chloride in 92 ml of tetrahydrofuran, and after 15 minutes, the solution of 5.1 g (maximum 26.2 mmol) of the compound, produced according to Example 2c, in 240 ml of tetrahydrofuran is added in drops. It is stirred for 2 more hours at −70° C., poured into a saturated ammonium chloride solution and extracted several times with ethyl acetate. The combined organic extracts are washed with water and saturated sodium chloride solution, dried on sodium sulfate, and the residue that is obtained after filtration and removal of the solvent is purified by chromatography on fine silica gel with a mobile solvent mixture that consists of n-hexane and ethyl acetate. 8.22 g (19.3 mmol, 73.5%) of title compound A, 0.706 g (1.65 mmol, 6.3%) of title compound B as well as 10.2 g of (4S)-4-(2-methyl-3-oxo-hept-6-en-2-yl)-2,2-dimethyl-1,3-dioxane are isolated in each case as a colorless oil. ¹H-NMR (CDCl₃) of A: δ=0.87 (3H), 0.96 (3H), 1.10 (1H), 1.23 (3H), 1.31 (6H), 1.37 (3H), 1.22-1.40 (2H), 1.43-1.84 (6H), 2.23 (1H), 2.43 (1H), 2.94 (1H), 3.34 (1H), 3.48 (1H), 3.81-4.01 (6H), 4.13 (1H), 4.97 (1H), 5.03 (1H), 5.71 (1H) ppm.

¹H-NMR (CDCl₃) of B: δ=0.88 (1H), 0.97 (3H), 1.07 (3H), 1.16 (3H), 1.31 (6H), 1.40 (3H), 1.08-1.75 (8H), 2.32 (1H), 2.42 (1H), 2.69 (1H), 3.31 (1H), 3.56 (1H), 3.81-4.00 (6H), 4.08 (1H), 4.98 (1H), 5.04 (1H), 5.72 (1H) ppm.

### EXAMPLE 2e

### (3S,6R,7S,8S)-4,4,8-Trimethyl-5-oxo-tridecane-1,3,7-triol

The solution of 6.78 g (15.9 mmol) of compound A, produced according to Example 2d, in a mixture that consists of 200 ml of ethanol and 50 ml of water is mixed with 6.7 g of p-toluenesulfonic acid-monohydrate, and it is stirred for 4 hours at 23° C. It is mixed with saturated sodium bicarbonate solution, extracted with dichloromethane, and the combined organic extracts are dried on sodium sulfate. The residue that is obtained after filtration and removal of the solvent is further reacted without purification. 5.8 g (maximum 15.9 mmol) of the title compound is isolated as a colorless oil.

¹H-NMR (CDCl₃): δ=0.91 (3H), 1.06 (3H), 1.14 (1H), 1.21 (3H), 1.48 (1H), 1.57-1.74 (4H), 2.13 (3H), 2.26 (1H), 2.39-2.56 (4H), 2.92 (1H), 3.28 (1H), 3.32 (1H), 3.41 (1H), 3.72 (1H), 3.81-3.94 (2H), 4.04 (1H), 4.99 (1H), 5.05 (1H), 5.70 (1H) ppm.

### EXAMPLE 2f

### (3S,6R,7S,8S)-4,4,8-Trimethyl-1,3,7-tris-(tert.-butyldimethylsilyloxy)-tridecan-5-one

The solution of 5.8 g (maximum 15.9 mmol) of the compound, produced according to Example 2e, in 240 ml of dichloromethane, is mixed at −70° C. under an atmosphere of dry argon with 22 ml of 2,6-lutidine, 21.7 ml of trifluoromethane-sulfonic acid-tert.butyldimethylsilyl ester, and within 15 hours, it is allowed to heat to 23° C. It is poured into saturated sodium bicarbonate solution, extracted with dichloromethane, the combined organic extracts are washed with 1N hydrochloric acid and saturated sodium bicarbonate solution and dried on sodium sulfate. The residue that is obtained after filtration and removal of the solvent is purified by chromatography on fine silica gel with a mobile solvent mixture that consists of n-hexane and ethyl acetate. 4.77 g (6.96 mmol, 43.8%) of the title compound as well as 4.37 g of an isomer mixture of silyl enol ether of the title compound, which can be converted into the title compound by treatment with tetrabutylammonium fluoride in tetrahydrofuran, are isolated.

¹H-NMR (CDCl₃): δ=0.01-0.10 (18H), 0.89 (27H), 0.95 (3H), 1.05 (3H), 1.09 (1H), 1.20 (3H), 1.33-1.76 (6H), 2.14 (3H), 2.26 (1H), 2.35-2.50 (3H), 3.13 (1H), 3.55 (1H), 3.65 (1H), 3.76 (1H), 3.86 (1H), 4.94 (1H), 4.99 (1H), 5.75 (1H) ppm.

### EXAMPLE 2g

### (3S,6R,7S,8S,12E/Z,15S)-4,4,8,12-Tetramethyl-6-allyl-1,3,7,15-tetrakis-(tert.-butyldimethylsilyloxy)-15-(2-methylbenzothiazol-5-yl)-pentadec-12-en-5-one

The solution of 7.2 g of [(3S)-3-(2-methylbenzothiazol-5-yl)-3-(tert.-butyldimethylsilyloxy)-propyl]-triphenylphosphonium iodide, which was produced analogously to the process described in WO 99/07692, WO 99049154, and WO 00/01333, in 50 ml of anhydrous tetrahydrofuran is mixed at 0° C. with 10.2 ml of a 1 molar solution of sodium hexamethyldisilazane in tetrahydrofuran and then with the solution of 4.73 g (6.90 mmol) of the compound, produced according to Example 2f, in 50 ml of tetrahydrofuran. It is allowed to react for 5 hours at 23° C., poured into a saturated ammonium chloride solution and extracted several times with ethyl acetate. The combined organic extracts are washed with water and saturated sodium chloride solution, dried on sodium sulfate, and the residue that is obtained after filtration and removal of the solvent is purified by chromatography on fine silica gel with a mobile solvent mixture that consists of n-hexane and ethyl acetate. 6.18 g (6.25 mmol, 90.6%) of the title compound is isolated as a colorless oil.

¹H-NMR (CDCl₃): δ=−0.13-0.08 (24H), 0.88 (36H), 0.89-1.67 (19H), 1.91 (2H), 2.19-2.52 (4H), 2.83 (3H), 3.13 (1H), 3.49-3.71 (2H), 3.75 (1H), 3.85 (1H), 4.74 (1H), 4.92 (1H), 4.98 (1H), 5.15 (1H), 5.75 (1H), 7.32 (1H), 7.73 (1H), 7.86 (1H) ppm.

Analogously to Example 1, the following AB fragments were produced:

### EXAMPLE 3

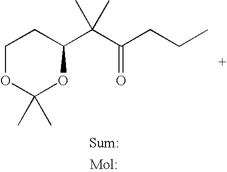

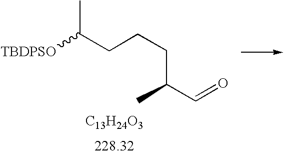

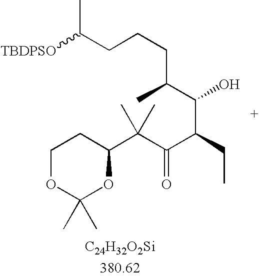

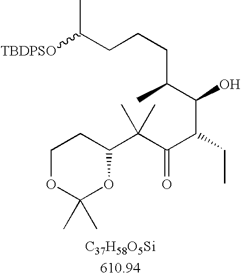

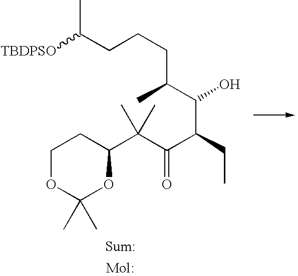

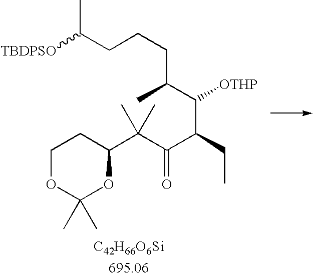

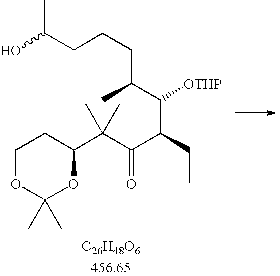

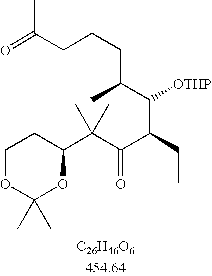

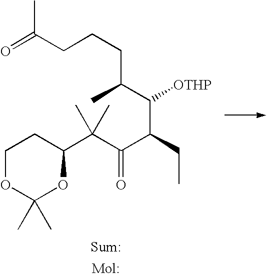

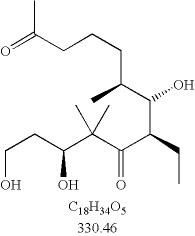

### EXAMPLE 4

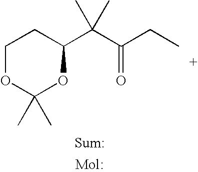

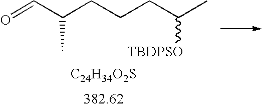

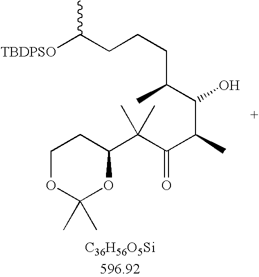

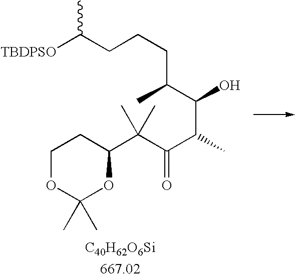

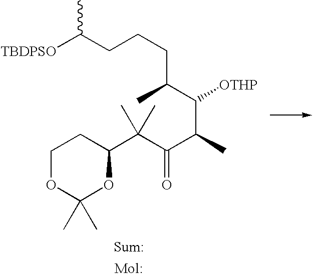

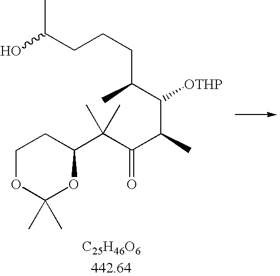

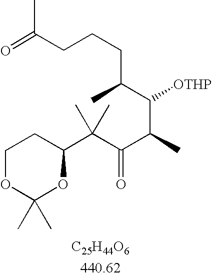

### EXAMPLE 5

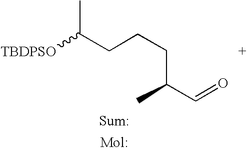

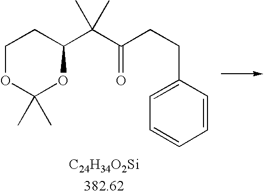

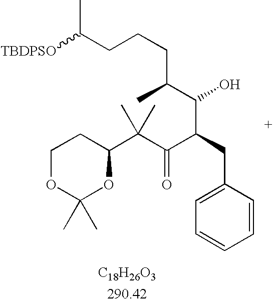

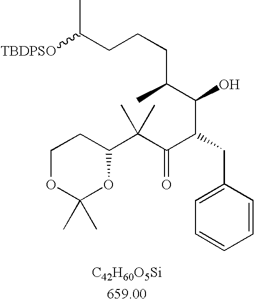

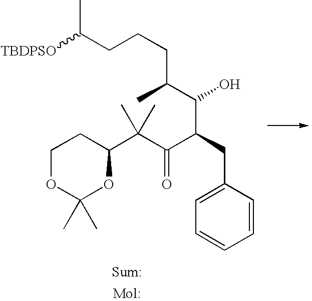

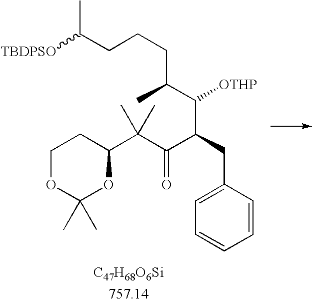

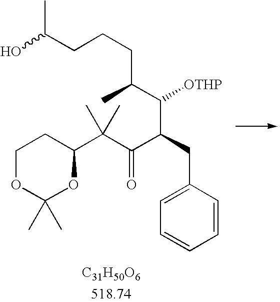

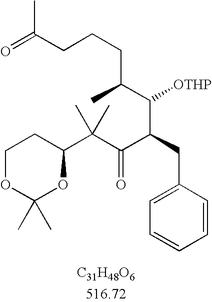

### EXAMPLE 6

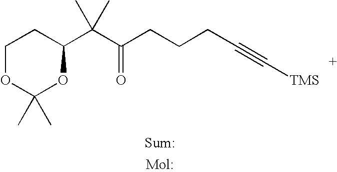

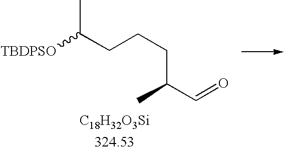

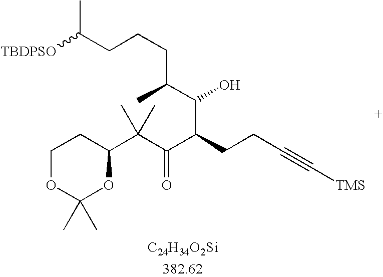

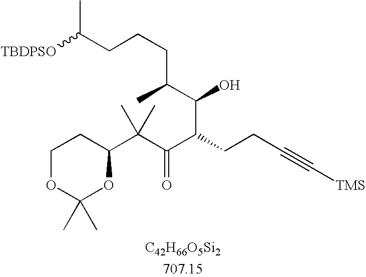

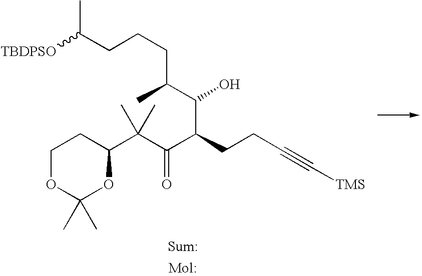

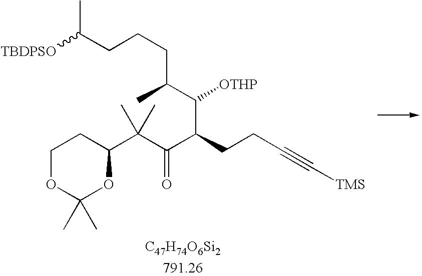

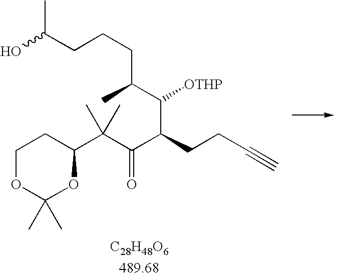

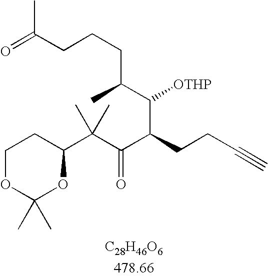

### EXAMPLE 7

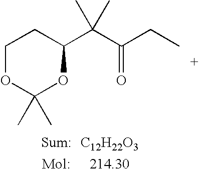

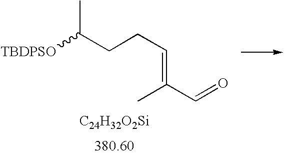

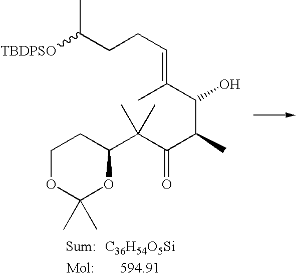

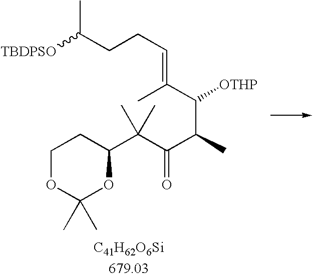

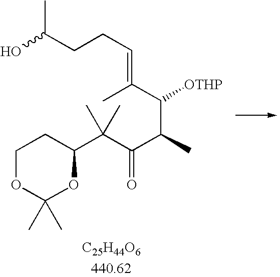

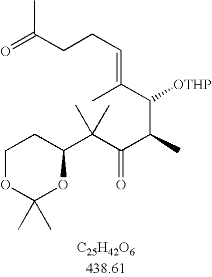

The methyl ketones of the thus produced AB-components can then be combined with the Wittig salts of a wide variety of C-components and converted into the especially preferred active ingredients:

(4S,7R,8S,9S,13Z,16S(E))-4,8-Dihydroxy-5,5,7,9,13-pentamethyl-16-[1-methyl-2-(2-methyl-thiazol-4-yl)-vinyl]-oxacyclohexadec-13-ene-2,6-dione

(4S,7R,8S,9S,13Z,16S(E))-4,8-Dihydroxy-16-[2-(2-hydroxymethyl-thiazol-4-yl)-1-methyl-vinyl]-5,5,7,9,13-pentamethyl-oxacyclohexadec-13-ene-2,6-dione

(4S,7R,8S,9S,13Z,16S(E))-16-[2-(2-Aminomethyl-thiazol-4-yl)-1-methyl-vinyl]-4,8-dihydroxy-5,5,7,9,13-pentamethyl-oxacyclohexadec-13-ene-2,6-dione

(1S,3S(E),7S,10R,11S,12S,16R)-7,11-Dihydroxy-8,8,10,12,16-pentamethyl-3-[1-methyl-2-(2-methyl-thiazol-4-yl)-vinyl]-4,17-dioxa-bicyclo[14.1.0]heptadecane-5,9-dione

(1S,3S(E),7S,10R,11S,12S,16R)-7,11-Dihydroxy-3-[2-(2-hydroxymethyl-thiazol-4-yl)-1-methyl-vinyl]-8,8,10,12,16-pentamethyl-4,17-dioxa-bicyclo[14.1.0]heptadecane-5,9-dione

(1S,3S(E),7S,10R,11S,12S,16R)-3-[2-(2-Aminomethyl-thiazol-4-yl)-1-methyl-vinyl]-7,11-dihydroxy-8,8,10,12,16-pentamethyl-4,17-dioxa-bicyclo[14.1.0]heptadecane-5,9-dione

(4S,7R,8S,9S,13Z,16S(E))-4,8-Dihydroxy-7-ethyl-5,5,9,13-tetramethyl-16-[1-methyl-2-(2-methyl-thiazol-4-yl)-vinyl]-oxacyclohexadec-13-ene-2,6-dione

(4S,7R,8S,9S,13Z,16S(E))-4,8-Dihydroxy-16-[2-(2-hydroxymethyl-thiazol-4-yl)-1-methyl-vinyl]-7-ethyl-5,5,9,13-tetramethyl-oxacyclohexadec-13-ene-2,6-dione

(4S,7R,8S,9S,13Z,16S(E))-16-[2-(2-Aminomethyl-thiazol-4-yl)-1-methyl-vinyl]-4,8-dihydroxy-7-ethyl-5,5,9,13-tetramethyl-oxacyclohexadec-13-ene-2,6-dione

(1S,3S(E),7S,10R,11S,12S,16R)-7,11-Dihydroxy-10-ethyl-8,8,12,16-tetramethyl-3-[1-methyl-2-(2-methyl-thiazol-4-yl)-vinyl]-4,17-dioxa-bicyclo[14.1.0]heptadecane-5,9-dione

(1S,3S(E),7S,10R,11S,12S,16R)-7,11-Dihydroxy-3-[2-(2-hydroxymethyl-thiazol-4-yl)-1-methyl-vinyl]-10-ethyl-8,8,12,16-tetramethyl-4,17-dioxa-bicyclo[14.1.0]-heptadecane-5,9-dione

(1S,3S(E),7S,10R,11S,12S,16R)-3-[2-(2-Aminomethyl-thiazol-4-yl)-1-methyl-vinyl]-7,11-dihydroxy-10-ethyl-8,8,12,16-tetramethyl-4,17-dioxa-bicyclo[14.1.0]-heptadecane-5,9-dione

(4S,7R,8S,9S,13Z,16S(Z))-4,8-Dihydroxy-5,5,7,9,13-pentamethyl-16-[1-fluoro-2-(2-methyl-thiazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-thiazol-4-yl)-1-fluoro-vinyl]-5,5,7,9,13-pentamethyl-oxacyclohexadec-13-ene-2,6-dione

(4S,7R,8S,9S,13Z,16S(Z))-16-[2-(2-Aminomethyl-thiazol-4-yl)-1-fluoro-vinyl]-4,8-dihydroxy-5,5,7,9,13-pentamethyl-oxacyclohexadec-13-ene-2,6-dione

(1S,3S(Z),7S,10R,11S,12S,16R)-7,11-Dihydroxy-8,8,10,12,16-pentamethyl-3-[1-fluoro-2-(2-methyl-thiazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-thiazol-4-yl)-1-fluoro-vinyl]-8,8,10,12,16-pentamethyl-4,17-dioxa-bicyclo[14.1.0]heptadecane-5,9-dione

(1S,3S(Z),7S,10R,11S,12S,16R)-3-[2-(2-Aminomethyl-thiazol-4-yl)-1-fluoro-vinyl]-7,11-dihydroxy-8,8,10,12,16-pentamethyl-4,17-dioxa-bicyclo[14.1.0]heptadecane-5,9-dione

(4S,7R,8S,9S,13Z,16S(Z))-4,8-Dihydroxy-5,5,7,9,13-pentamethyl-16-[1-chloro-2-(2-methyl-thiazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-thiazol-4-yl)-1-chloro-vinyl]-5,5,7,9,13-pentamethyl-oxacyclohexadec-13-ene-2,6-dione

(4S,7R,8S,9S,13Z,16S(Z))-16-[2-(2-Aminomethyl-thiazol-4-yl)-1-chloro-vinyl]-4,8-dihydroxy-5,5,7,9,13-pentamethyl-oxacyclohexadec-13-ene-2,6-dione

(1S,3S(Z),7S,10R,11S,12S,16R)-7,11-Dihydroxy-8,8,10,12,16-pentamethyl-3-[1-chloro-2-(2-methyl-thiazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-thiazol-4-yl)-1-chloro-vinyl]-8,8,10,12,16-pentamethyl-4,17-dioxa-bicyclo[14.1.0]heptadecane-5,9-dione

(1S,3S(Z),7S,10R,11S,12S,16R)-3-[2-(2-Aminomethyl-thiazol-4-yl)-1-chloro-vinyl]-7,11-dihydroxy-8,8,10,12,16-pentamethyl-4,17-dioxa-bicyclo[14.1.0]heptadecane-5,9-dione

(4S,7R,8S,9S,13Z,16S(Z))-4,8-Dihydroxy-7-ethyl-5,5,9,13-tetramethyl-16-[1-fluoro-2-(2-methyl-thiazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-thiazol-4-yl)-1-fluoro-vinyl]-7-ethyl-5,5,9,13-tetramethyl-oxacyclohexadec-13-ene-2,6-dione

(4S,7R,8S,9S,13Z,16S(Z))-16-[2-(2-Aminomethyl-thiazol-4-yl)-1-fluoro-vinyl]-4,8-dihydroxy-7-ethyl-5,5,9,13-tetramethyl-oxacyclohexadec-13-ene-2,6-dione

(1S,3S(Z),7S,10R,11S,12S,16R)-7,11-Dihydroxy-10-ethyl-8,8,12,16-tetramethyl-3-[1-fluoro-2-(2-methyl-thiazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-thiazol-4-yl)-1-fluoro-vinyl]-10-ethyl-8,8,12,16-tetramethyl-4,17-dioxa-bicyclo[14.1.0]-heptadecane-5,9-dione

(1S,3S(Z),7S,10R,11S,12S,16R)-3-[2-(2-Aminomethyl-thiazol-4-yl)-1-fluoro-vinyl]-7,11-dihydroxy-10-ethyl-8,8,12,16-tetramethyl-4,17-dioxa-bicyclo[14.1.0]-heptadecane-5,9-dione

(4S,7R,8S,9S,13Z,16S(Z))-4,8-Dihydroxy-7-ethyl-5,5,9,13-tetramethyl-16-[1-chloro-2-(2-methyl-thiazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-thiazol-4-yl)-1-chloro-vinyl]-7-ethyl-5,5,9,13-tetramethyl-oxacyclohexadec-13-ene-2,6-dione

(4S,7R,8S,9S,13Z,16S(Z))-16-[2-(2-Aminomethyl-thiazol-4-yl)-1-chloro-vinyl]-4,8-dihydroxy-7-ethyl-5,5,9,13-tetramethyl-oxacyclohexadec-13-ene-2,6-dione

(1S,3S(Z),7S,10R,11S,12S,16R)-7,11-Dihydroxy-10-ethyl-8,8,12,16-tetramethyl-3-[1-chloro-2-(2-methyl-thiazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-thiazol-4-yl)-1-chloro-vinyl]-10-ethyl-8,8,12,16-tetramethyl-4,17-dioxa-bicyclo[14.1.0]-heptadecane-5,9-dione

(1S,3S(Z),7S,10R,11S,12S,16R)-3-[2-(2-Aminomethyl-thiazol-4-yl)-1-chloro-vinyl]-7,11-dihydroxy-10-ethyl-8,8,12,16-tetramethyl-4,17-dioxa-bicyclo[14.1.0]-heptadecane-5,9-dione

(4S,7R,8S,9S,13Z,16S(E))-4,8-Dihydroxy-5,5,7,9,13-pentamethyl-16-[1-methyl-2-(2-pyridyl)-vinyl]-oxacyclohexadec-13-ene-2,6-dione

(1S,3S(E),7S,10R,11S,12S,16R)-7,11-Dihydroxy-8,8,10,12,16-pentamethyl-3-[1-methyl-2-(2-pyridyl)-vinyl]-4,17-dioxa-bicyclo[14.1.0]heptadecane-5,9-dione

(4S,7R,8S,9S,13Z,16S(E))-4,8-Dihydroxy-7-ethyl-5,5,9,13-tetramethyl-16-[1-methyl-2-(2-pyridyl)-vinyl]-oxacyclohexadec-13-ene-2,6-dione

(1S,3S(E),7S,10R,11S,12S,16R)-7,11-Dihydroxy-10-ethyl-8,8,12,16-tetramethyl-3-[1-methyl-2-(2-pyridyl)-vinyl]-4,17-dioxa-bicyclo[14.1.0]heptadecane-5,9-dione

(4S,7R,8S,9S,13Z,16S(Z))-4,8-Dihydroxy-5,5,7,9,13-pentamethyl-16-[1-fluoro-2-(2-pyridyl)-vinyl]-oxacyclohexadec-13-ene-2,6-dione

(1S,3S(Z),7S,10R,11S,12S,16R)-7,11-Dihydroxy-8,8,10,12,16-pentamethyl-3-[1-fluoro-2-(2-pyridyl)-vinyl]-4,17-dioxa-bicyclo[14.1.0]heptadecane-5,9-dione

(4S,7R,8S,9S,13Z,16S(Z))-4,8-Dihydroxy-5,5,7,9,13-pentamethyl-16-[1-chloro-2-(2-pyridyl)-vinyl]-oxacyclohexadec-13-ene-2,6-dione

(1S,3S(Z),7S,10R,11S,12S,16R)-7,11-Dihydroxy-8,8,10,12,16-pentamethyl-3-[1-chloro-2-(2-pyridyl)-vinyl]-4,17-dioxa-bicyclo[14.1.0]heptadecane-5,9-dione

(4S,7R,8S,9S,13Z,16S(Z))-4,8-Dihydroxy-7-ethyl-5,5,9,13-tetramethyl-16-[1-fluoro-2-(2-pyridyl)-vinyl]-oxacyclohexadec-13-ene-2,6-dione

(1S,3S(Z),7S,10R,11S,12S,16R)-7,11-Dihydroxy-10-ethyl-8,8,12,16-tetramethyl-3-[1-fluoro-2-(2-pyridyl)-vinyl]-4,17-dioxa-bicyclo[14.1.0]heptadecane-5,9-dione

(4S,7R,8S,9S,13Z,16S(Z))-4,8-Dihydroxy-7-ethyl-5,5,9,13-tetramethyl-16-[1-chloro-2-(2-pyridyl)-vinyl]-oxacyclohexadec-13-ene-2,6-dione

(1S,3S(Z),7S,10R,11S,12S,16R)-7,11-Dihydroxy-10-ethyl-8,8,12,16-tetramethyl-3-[1-chloro-2-(2-pyridyl)-vinyl]-4,17-dioxa-bicyclo[14.1.0]heptadecane-5,9-dione

(4S,7R,8S,9S,13Z,16S(E))-4,8-Dihydroxy-5,5,7,9,13-pentamethyl-16-[1-methyl-2-(2-methyl-oxazol-4-yl)-vinyl]-oxacyclohexadec-13-ene-2,6-dione

(4S,7R,8S,9S,13Z,16S(E))-4,8-Dihydroxy-16-[2-(2-hydroxymethyl-oxazol-4-yl)-1-methyl-vinyl]-5,5,7,9,13-pentamethyl-oxacyclohexadec-13-ene-2,6-dione

(4S,7R,8S,9S,13Z,16S(E))-16-[2-(2-Aminomethyl-oxazol-4-yl)-1-methyl-vinyl]-4,8-dihydroxy-5,5,7,9,13-pentamethyl-oxacyclohexadec-13-ene-2,6-dione

(1S,3S(E),7S,10R,11S,12S,16R)-7,11-Dihydroxy-8,8,10,12,16-pentamethyl-3-[1-methyl-2-(2-methyl-oxazol-4-yl)-vinyl]-4,17-dioxa-bicyclo[14.1.0]heptadecane-5,9-dione

(1S,3S(E),7S,10R,11S,12S,16R)-7,11-Dihydroxy-3-[2-(2-hydroxymethyl-oxazol-4-yl)-1-methyl-vinyl]-8,8,10,12,16-pentamethyl-4,17-dioxa-bicyclo[14.1.0]heptadecane-5,9-dione

(1S,3S(E),7S,10R,11S,12S,16R)-3-[2-(2-Aminomethyl-oxazol-4-yl)-1-methyl-vinyl]-7,11-dihydroxy-8,8,10,12,16-pentamethyl-4,17-dioxa-bicyclo[14.1.0]heptadecane-5,9-dione

(4S,7R,8S,9S,13Z,16S(E))-4,8-Dihydroxy-7-ethyl-5,5,9,13-tetramethyl-16-[1-methyl-2-(2-methyl-oxazol-4-yl)-vinyl]-oxacyclohexadec-13-ene-2,6-dione

(4S,7R,8S,9S,13Z,16S(E))-4,8-Dihydroxy-16-[2-(2-hydroxymethyl-oxazol-4-yl)-1-methyl-vinyl]-7-ethyl-5,5,9,13-tetramethyl-oxacyclohexadec-13-ene-2,6-dione

(4S,7R,8S,9S,13Z,16S(E))-16-[2-(2-Aminomethyl-oxazol-4-yl)-1-methyl-vinyl]-4,8-dihydroxy-7-ethyl-5,5,9,13-tetramethyl-oxacyclohexadec-13-ene-2,6-dione

(1S,3S(E),7S,10R,11S,12S,16R)-7,11-Dihydroxy-10-ethyl-8,8,12,16-tetramethyl-3-[1-methyl-2-(2-methyl-oxazol-4-yl)-vinyl]-4,17-dioxa-bicyclo[14.1.0]heptadecane-5,9-dione

(1S,3S(E),7S,10R,11S,12S,16R)-7,11-Dihydroxy-3-[2-(2-hydroxymethyl-oxazol-4-yl)-1-methyl-vinyl]-10-ethyl-8,8,12,16-tetramethyl-4,17-dioxa-bicyclo[14.1.0]-heptadecane-5,9-dione

(1S,3S(E),7S,10R,11S,12S,16R)-3-[2-(2-Aminomethyl-oxazol-4-yl)-1-methyl-vinyl]-7,11-dihydroxy-10-ethyl-8,8,12,16-tetramethyl-4,17-dioxa-bicyclo[14.1.0]-heptadecane-5,9-dione

(4S,7R,8S,9S,13Z,16S(Z))-4,8-Dihydroxy-5,5,7,9,13-pentamethyl-16-[1-fluoro-2-(2-methyl-oxazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-oxazol-4-yl)-1-fluoro-vinyl]-5,5,7,9,13-pentamethyl-oxacyclohexadec-13-ene-2,6-dione

(4S,7R,8S,9S,13Z,16S(Z))-16-[2-(2-Aminomethyl-oxazol-4-yl)-1-fluoro-vinyl]-4,8-dihydroxy-5,5,7,9,13-pentamethyl-oxacyclohexadec-13-ene-2,6-dione

(1S,3S(Z),7S,10R,11S,12S,16R)-7,11-Dihydroxy-8,8,10,12,16-pentamethyl-3-[1-fluoro-2-(2-methyl-oxazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-oxazol-4-yl)-1-fluoro-vinyl]-8,8,10,12,16-pentamethyl-4,17-dioxa-bicyclo[14.1.0]heptadecane-5,9-dione

(1S,3S(Z),7S,10R,11S,12S,16R)-3-[2-(2-Aminomethyl-oxazol-4-yl)-1-fluoro-vinyl]-7,11-dihydroxy-8,8,10,12,16-pentamethyl-4,17-dioxa-bicyclo[14.1.0]heptadecane-5,9-dione

(4S,7R,8S,9S,13Z,16S(Z))-4,8-Dihydroxy-5,5,7,9,13-pentamethyl-16-[1-chloro-2-(2-methyl-oxazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-oxazol-4-yl)-1-chloro-vinyl]-5,5,7,9,13-pentamethyl-oxacyclohexadec-13-ene-2,6-dione

(4S,7R,8S,9S,13Z,16S(Z))-16-[2-(2-Aminomethyl-oxazol-4-yl)-1-chloro-vinyl]-4,8-dihydroxy-5,5,7,9,13-pentamethyl-oxacyclohexadec-13-ene-2,6-dione

(1S,3S(Z),7S,10R,11S,12S,16R)-7,11-Dihydroxy-8,8,10,12,16-pentamethyl-3-[1-chloro-2-(2-methyl-oxazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-oxazol-4-yl)-1-chloro-vinyl]-8,8,10,12,16-pentamethyl-4,17-dioxa-bicyclo[14.1.0]heptadecane-5,9-dione (1S,3S(Z),7S,10R,11S,12S,16R)-3-[2-(2-Aminomethyl-oxazol-4-yl)-1-chloro-vinyl]-7,11-dihydroxy-8,8,10,12,16-pentamethyl-4,17-dioxa-bicyclo[14.1.0]heptadecane-5,9-dione

(4S,7R,8S,9S,13Z,16S(Z))-4,8-Dihydroxy-7-ethyl-5,5,9,13-tetramethyl-16-[1-fluoro-2-(2-methyl-oxazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-oxazol-4-yl)-1-fluoro-vinyl]-7-ethyl-5,5,9,13-tetramethyl-oxacyclohexadec-13-ene-2,6-dione

(4S,7R,8S,9S,13Z,16S(Z))-16-[2-(2-Aminomethyl-oxazol-4-yl)-1-fluoro-vinyl]-4,8-dihydroxy-7-ethyl-5,5,9,13-tetramethyl-oxacyclohexadec-13-ene-2,6-dione

(1S,3S(Z),7S,10R,11S, 12S,16R)-7,11-Dihydroxy-10-ethyl-8,8,12,16-tetramethyl-3-[1-fluoro-2-(2-methyl-oxazol4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-oxazol-4-yl)-1-fluoro-vinyl]-10-ethyl-8,8,12,16-tetramethyl-4,17-dioxa-bicyclo[14.1.0]-heptadecane-5,9-dione

(1S,3S(Z),7S,10R,11S,12S,16R)-3-[2-(2-Aminomethyl-oxazol-4-yl)-1-fluoro-vinyl]-7,11-dihydroxy-10-ethyl-8,8,12,16-tetramethyl-4,17-dioxa-bicyclo[14.1.0]-heptadecane-5,9-dione

(4S,7R,8S,9S,13Z,16S(Z))-4,8-Dihydroxy-7-ethyl-5,5,9,13-tetramethyl-16-[1-chloro-2-(2-methyl-oxazol-4-yl)-vinyl]-oxacyclohexadec-13-ene-2,6-dione

(4S,7R,8S,9S,13Z,16S(Z))-4,8-Dihydroxy-16-[2-(2-hydroxymethyl-oxazol-4-yl)-1-chloro-vinyl]-7-ethyl-5,5,9,13-tetramethyl-oxacyclohexadec-13-ene-2,6-dione

(4S,7R,8S,9S,13Z,16S(Z))-16-[2-(2-Aminomethyl-oxazol-4-yl)-1-chloro-vinyl]-4,8-dihydroxy-7-ethyl-5,5,9,13-tetramethyl-oxacyclohexadec-13-ene-2,6-dione

(1S,3S(Z),7S,10R,11S,12S,16R)-7,11-Dihydroxy-10-ethyl-8,8,12,16-tetramethyl-3-[1-chloro-2-(2-methyl-oxazol-4-yl)-vinyl]-4,17-dioxa-bicyclo[14.1.0]heptadecane-5,9-dione

(1S,3S(Z),7S,10R,11S,12S,16R)-7,11-Dihydroxy-3-[2-(2-hydroxymethyl-oxazol-4-yl)-1-chloro-vinyl]-10-ethyl-8,8,12,16-tetramethyl-4,17-dioxa-bicyclo[14.1.0]-heptadecane-5,9-dione

(1S,3S(Z),7S,10R,11S,12S,16R)-3-[2-(2-Aminomethyl-oxazol-4-yl)-1-chloro-vinyl]-7,11-dihydroxy-10-ethyl-8,8,12,16-tetramethyl-4,17-dioxa-bicyclo[14.1.0]-heptadecane-5,9-dione

(4S,7R,8S,9S,13Z,16S(E))-4,8-Dihydroxy-5,5,7,9,13-pentamethyl-16-[2-(2-methyl-thiazol-4-yl)-vinyl]-oxacyclohexadec-13-ene-2,6-dione

(4S,7R,8S,9S,13Z,16S(E))-4,8-Dihydroxy-16-[2-(2-hydroxymethyl-thiazol-4-yl)-vinyl]-5,5,7,9,13-pentamethyl-oxacyclohexadec-13-ene-2,6-dione (4S,7R,8S,9S,13Z,16S(E))-16-[2-(2-Aminomethyl-thiazol-4-yl)-vinyl]-4,8-dihydroxy-5,5,7,9,13-pentamethyl-oxacyclohexadec-13-ene-2,6-dione (1S,3S(E),7S,10R,11S,12S,16R)-7,11-Dihydroxy-8,8,10,12,16-pentamethyl-3-[2-(2-methyl-thiazol-4-yl)-vinyl]-4,17-dioxa-bicyclo[14.1.0]heptadecane-5,9-dione (1S,3S(E),7S,10R,11S,12S,16R)-7,11-Dihydroxy-3-[2-(2-hydroxymethyl-thiazol-4-yl)-vinyl]-8,8,10,12,16-pentamethyl-4,17-dioxa-bicyclo[14.1.0]heptadecane-5,9-dione

(1S,3S(E),7S,10R,11S,12S,16R)-3-[2-(2-Aminomethyl-thiazol-4-yl)-vinyl]-7,11-dihydroxy-8,8,10,12,16-pentamethyl-4,17-dioxa-bicyclo[14.1.0]heptadecane-5,9-dione

(4S,7R,8S,9S,13Z,16S(E))-4,8-Dihydroxy-7-ethyl-5,5,9,13-tetramethyl-16-[2-(2-methyl-thiazol-4-yl)-vinyl]-oxacyclohexadec-13-ene-2,6-dione

(4S,7R,8S,9S,13Z,16S(E))-4,8-Dihydroxy-16-[2-(2-hydroxymethyl-thiazol-4-yl)-vinyl]-7-ethyl-5,5,9,13-tetramethyl-oxacyclohexadec-13-ene-2,6-dione

(4S,7R,8S,9S,13Z,16S(E))-16-[2-(2-Aminomethyl-thiazol-4-yl)-vinyl]-4,8-dihydroxy-7-ethyl-5,5,9,13-tetramethyl-oxacyclohexadec-13-ene-2,6-dione

(1S,3S(E),7S,10R,11S,12S,16R)-7,11-Dihydroxy-10-ethyl-8,8,12,16-tetramethyl-3-[2-(2-methyl-thiazol-4-yl)-vinyl]-4,17-dioxa-bicyclo[14.1.0]heptadecane-5,9-dione

(1S,3S(E),7S,10R,11S,12S,16R)-7,11-Dihydroxy-3-[2-(2-hydroxymethyl-thiazol-4-yl)-vinyl]-10-ethyl-8,8,12,16-tetramethyl-4,17-dioxa-bicyclo[14.1.0]heptadecane-5,9-dione

(1S,3S(E),7S,10R,11S,12S,16R)-3-[2-(2-Aminomethyl-thiazol-4-yl)-vinyl]-7,11-dihydroxy-10-ethyl-8,8,12,16-tetramethyl-4,17-dioxa-bicyclo[14.1.0]heptadecane-5,9-dione

(4S,7R,8S,9S,13Z,16S(E))-4,8-Dihydroxy-5,5,7,9,13-pentamethyl-16-[2-(2-pyridyl)-vinyl]-oxacyclohexadec-13-ene-2,6-dione

(1S,3S(E),7S,10R,11S,12S,16R)-7,11-Dihydroxy-8,8,10,12,16-pentamethyl-3-[2-(2-pyridyl)-vinyl]-4,17-dioxa-bicyclo[14.1.0]heptadecane-5,9-dione

(4S,7R,8S,9S,13Z,16S(E))-4,8-Dihydroxy-7-ethyl-5,5,9,13-tetramethyl-16-[2-(2-pyridyl)-vinyl]-oxacyclohexadec-13-ene-2,6-dione

(1S,3S(E),7S,10

R,11S,12S,16R)-7,11-Dihydroxy-10-ethyl-8,8,12,16-tetramethyl-3-[2-(2-pyridyl)-vinyl]-4,17-dioxa-bicyclo[14.1.0]heptadecane-5,9-dione

(4S,7R,8S,9S,13Z,16S)-4,8-Dihydroxy-5,5,7,9,13-pentamethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-5,5,7,9,13-pentamethyl-oxacyclohexadec-13-ene-2,6-dione

(4S,7R,8S,9S,13Z,16S)-16-(2-Aminomethyl-benzothiazol-5-yl)-4,8-dihydroxy-5,5,7,9,13-pentamethyl-oxacyclohexadec-13-ene-2,6-dione

(1S,3S,7S,10R,11S,12S,16R)-7,11-Dihydroxy-8,8,10,12,16-pentamethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-8,8,10,12,16-pentamethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-8,8,10,12,16-pentamethyl-4,17-dioxa-bicyclo[14.1.0]heptadecane-5,9-dione

(4S,7R,8S,9S,13Z,16S)-4,8-Dihydroxy-7-ethyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-ethyl-5,5,9,13-tetramethyl-oxacyclohexadec-13-ene-2,6-dione

(4S,7R,8S,9S,13Z,16S)-16-(2-Aminomethyl-benzothiazol-5-yl)-4,8-dihydroxy-7-ethyl-5,5,9,13-tetramethyl-oxacyclohexadec-13-ene-2,6-dione

(1S,3S,7S,10R,11S,12S,16R)-7,11-Dihydroxy-10-ethyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-ethyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-ethyl-8,8,12,16-tetramethyl-4,17-dioxa-bicyclo[14.1.0]heptadecane-5,9-dione

(4S,7R,8S,9S,13Z,16S)-4,8-Dihydroxy-7-propyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-propyl-5,5,9,13-tetramethyl-oxacyclohexadec-13-ene-2,6-dione

(4S,7R,8S,9S,13Z,16S)-16-(2-Aminomethyl-benzothiazol-5-yl)-4,8-dihydroxy-7-propyl-5,5,9,13-tetramethyl-oxacyclohexadec-13-ene-2,6-dione

(1S,3S,7S,10R,11S,12S,16R)-7,11-Dihydroxy-10-propyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-propyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-propyl-8,8,12,16-tetramethyl-4,17-dioxa-bicyclo[14.1.0]heptadecane-5,9-dione

(4S,7R,8S,9S,13Z,16S)-4,8-Dihydroxy-7-butyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-butyl-5,5,9,13-tetramethyl-oxacyclohexadec-13-ene-2,6-dione

(4S,7R,8S,9S,13Z,16S)-16-(2-Aminomethyl-benzothiazol-5-yl)-4,8-dihydroxy-7-butyl-5,5,9,13-tetramethyl-oxacyclohexadec-13-ene-2,6-dione

(1S,3S,7S,10R,11S,12S,16R)-7,11-Dihydroxy-10-butyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-butyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-butyl-8,8,12,16-tetramethyl-4,17-dioxa-bicyclo[14.1.0]heptadecane-5,9-dione

(4S,7R,8S,9S,13Z,16S)-4,8-Dihydroxy-7-allyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-allyl-5,5,9,13-tetramethyl-oxacyclohexadec-13-ene-2,6-dione

(4S,7R,8S,9S,13Z,16S)-16-(2-Aminomethyl-benzothiazol-5-yl)-4,8-dihydroxy-7-allyl-5,5,9,13-tetramethyl-oxacyclohexadec-13-ene-2,6-dione

(1S,3S,7S,10R,11S,12S,16R)-7,11-Dihydroxy-10-allyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-allyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-allyl-8,8,12,16-tetramethyl-4,17-dioxa-bicyclo[14.1.0]heptadecane-5,9-dione

(4S,7R,8S,9S,13Z,16S)-4,8-Dihydroxy-7-prop-2-inyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-prop-2-inyl-5,5,9,13-tetramethyl-oxacyclohexadec-13-ene-2,6-dione

(4S,7R,8S,9S,13Z,16S)-16-(2-Aminomethyl-benzothiazol-5-yl)-4,8-dihydroxy-7-prop-2-inyl-5,5,9,13-tetramethyl-oxacyclohexadec-13-ene-2,6-dione

(1S,3S,7S,10R,11S,12S,16R)-7,11-Dihydroxy-10-prop-2-inyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-prop-2-inyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-prop-2-inyl-8,8,12,16-tetramethyl-4,17-dioxa-bicyclo[14.1.0]heptadecane-5,9-dione

(4S,7R,8S,9S,13Z,16S)-4,8-Dihydroxy-7-but-3-enyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-but-3-enyl-5,5,9,13-tetramethyl-oxacyclohexadec-13-ene-2,6-dione

(4S,7R,8S,9S,13Z,16S)-6-(2-Aminomethyl-benzothiazol-5-yl)-4,8-dihydroxy-7-but-3-enyl-5,5,9,13-tetramethyl-oxacyclohexadec-13-ene-2,6-dione

(1S,3S,7S,10R,11S,12S,16R)-7,11-Dihydroxy-10-but-3-enyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-but-3-enyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-but-3-enyl-8,8,12,16-tetramethyl-4,17-dioxa-bicyclo[14.1.0]heptadecane-5,9-dione

(4S,7R,8S,9S,13Z,16S)-4,8-Dihydroxy-7-but-3-inyl-5,5,9,13-tetramethyl-16-(2-methyl-benzothiazol-5-yl)-oxacyclohexadec-13-ene-2,6-dione

(4S,7R,8S,9S,13Z,16S)-4,8-Dihydroxy-16-(2-hydroxymethyl-benzothiazol-5-yl)-7-but-3-inyl-5,5,9,13-tetramethyl-oxacyclohexadec-13-ene-2,6-dione (4S,7R,8S,9S,13Z,16S)-16-(2-Aminomethyl-benzothiazol-5-yl)-4,8-dihydroxy-7-but-3-inyl-5,5,9,13-tetramethyl-oxacyclohexadec-13-ene-2,6-dione

(1S,3S,7S,10R,11S,12S,16R)-7,11-Dihydroxy-10-but-3-inyl-8,8,12,16-tetramethyl-3-(2-methyl-benzothiazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzothiazol-5-yl)-10-but-3-inyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzothiazol-5-yl)-7,11-dihydroxy-10-but-3-inyl-8,8,12,16-tetramethyl-4,17-dioxa-bicyclo[14.1.0]heptadecane-5,9-dione

(4S,7R,8S,9S,13Z,16S)-4,8-Dihydroxy-5,5,7,9,13-pentamethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-5,5,7,9,13-pentamethyl-oxacyclohexadec-13-ene-2,6-dione

(4S,7R,8S,9S,13Z,16S)-16-(2-Aminomethyl-benzoxazol-5-yl)-4,8-dihydroxy-5,5,7,9,13-pentamethyl-oxacyclohexadec-13-ene-2,6-dione

(1S,3S,7S,10R,11S,12S,16R)-7,11-Dihydroxy-8,8,10,12,16-pentamethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-8,8,10,12,16-pentamethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-8,8,10,12,16-pentamethyl-4,17-dioxa-bicyclo[14.1.0]heptadecane-5,9-dione

(4S,7R,8S,9S,13Z,16S)-4,8-Dihydroxy-7-ethyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-ethyl-5,5,9,13-tetramethyl-oxacyclohexadec-13-ene-2,6-dione

(4S,7R,8S,9S,13Z,16S)-16-(2-Aminomethyl-benzoxazol-5-yl)-4,8-dihydroxy-7-ethyl-5,5,9,13-tetramethyl-oxacyclohexadec-13-ene-2,6-dione

(1S,3S,7S,10R,11S,12S,16R)-7,11-Dihydroxy-10-ethyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-ethyl-8,8,12,16-tetramethyl-4,17-dioxa-bicyclo[14.1.0]heptadecane-5,9-dione

(1S,3 S,7S,10R,11S,12S,16R)-3-(2-Aminomethyl-benzoxazol-5-yl)-7,11-dihydroxy-10-ethyl-8,8,12,16-tetramethyl-4,17-dioxa-bicyclo[14.1.0]heptadecane-5,9-dione

(4S,7R,8S,9S,13Z,16S)-4,8-Dihydroxy-7-propyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-propyl-5,5,9,13-tetramethyl-oxacyclohexadec-13-ene-2,6-dione

(4S,7R,8S,9S,13Z,16S)-16-(2-Aminomethyl-benzoxazol-5-yl)-4,8-dihydroxy-7-propyl-5,5,9,13-tetramethyl-oxacyclohexadec-13-ene-2,6-dione

(1S,3 S,7S,10R,11S,12S,16R)-7,11-Dihydroxy-10-propyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-propyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-10-propyl-8,8,12,16-tetramethyl-4,17-dioxa-bicyclo[14.1.0]heptadecane-5,9-dione

(4S,7R,8S,9S,13Z,16S)-4,8-Dihydroxy-7-butyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-butyl-5,5,9,13-tetramethyl-oxacyclohexadec-13-ene-2,6-dione

(4S,7R,8S,9S,13Z,16S)-16-(2-Aminomethyl-benzoxazol-5-yl)-4,8-dihydroxy-7-butyl-5,5,9,13-tetramethyl-oxacyclohexadec-13-ene-2,6-dione

(1S,3S,7S,10R,11S,12S,16R)-7,11-Dihydroxy-10-butyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-butyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-10-butyl-8,8,12,16-tetramethyl-4,17-dioxa-bicyclo[14.1.0]heptadecane-5,9-dione

(4S,7R,8S,9S,13Z,16S)-4,8-Dihydroxy-7-allyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-allyl-5,5,9,13-tetramethyl-oxacyclohexadec-13-ene-2,6-dione (4S,7R,8S,9S,13Z,16S)-16-(2-Aminomethyl-benzoxazol-5-yl)-4,8-dihydroxy-7-allyl-5,5,9,13-tetramethyl-oxacyclohexadec-13-ene-2,6-dione

(1S,3S,7S,10R,11S,12S,16R)-7,11-Dihydroxy-10-allyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-allyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-10-allyl-8,8,12,16-tetramethyl-4,17-dioxa-bicyclo[14.1.0]heptadecane-5,9-dione

(4S,7R,8S,9S,13Z,16S)-4,8-Dihydroxy-7-prop-2-inyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-prop-2-inyl-5,5,9,13-tetramethyl-oxacyclohexadec-13-ene-2,6-dione

(4S,7R,8S,9S,13Z,16S)-16-(2-Aminomethyl-benzoxazol-5-yl)-4,8-dihydroxy-7-prop-2-inyl-5,5,9,13-tetramethyl-oxacyclohexadec-13-ene-2,6-dione

(1S,3S,7S,10R,11S,12S,16R)-7,11-Dihydroxy-10-prop-2-inyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-prop-2-inyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-10-prop-2-inyl-8,8,12,16-tetramethyl-4,17-dioxa-bicyclo[14.1.0]heptadecane-5,9-dione

(4S,7R,8S,9S,13Z,16S)-4,8-Dihydroxy-7-but-3-enyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-but-3-enyl-5,5,9,13-tetramethyl-oxacyclohexadec-13-ene-2,6-dione

(4S,7R,8S,9S,13Z,16S)-16-(2-Aminomethyl-benzoxazol-5-yl)-4,8-dihydroxy-7-but-3-enyl-5,5,9,13-tetramethyl-oxacyclohexadec-13-ene-2,6-dione

(1S,3S,7S,10R,11S,12S,16R)-7,11-Dihydroxy-10-but-3-enyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-but-3-enyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-10-but-3-enyl-8,8,12,16-tetramethyl-4,17-dioxa-bicyclo[14.1.0]heptadecane-5,9-dione

(4S,7R,8S,9S,13Z,16S)-4,8-Dihydroxy-7-but-3-inyl-5,5,9,13-tetramethyl-16-(2-methyl-benzoxazol-5-yl)-oxacyclohexadec-13-ene-2,6-dione

(4S,7R,8S,9S,13Z,16S)-4,8-Dihydroxy-16-(2-hydroxymethyl-benzoxazol-5-yl)-7-but-3-inyl-5,5,9,13-tetramethyl-oxacyclohexadec-13-ene-2,6-dione

(4S,7R,8S,9S,13Z,16S)-16-(2-Aminomethyl-benzoxazol-5-yl)-4,8-dihydroxy-7-but-3-inyl-5,5,9,13-tetramethyl-oxacyclohexadec-13-ene-2,6-dione

(1S,3S,7S,10R,11S,12S,16R)-7,11-Dihydroxy-10-but-3-inyl-8,8,12,16-tetramethyl-3-(2-methyl-benzoxazol-5-yl)-4,17-dioxa-bicyclo[14.1.0]heptadecane-5,9-dione

(1S,3S,7S,10R,11S,12S,16R)-7,11-Dihydroxy-3-(2-hydroxymethyl-benzoxazol-5-yl)-10-but-3-inyl-8,8,12,16-tetramethyl-4,17-dioxa-bicyclo[14.1.0]heptadecane-5,9-dione

(1S,3S,7S,10R,11S,12S,16R)-3-(2-Aminomethyl-benzoxazol-5-yl)-7,11-dihydroxy-10-but-3-inyl-8,8,12,16-tetramethyl-4,17-dioxa-bicyclo[14.1.0]heptadecane-5,9-dione

1. Process for the production of C₁-C₁₅-epothilone fragments of general formula I, 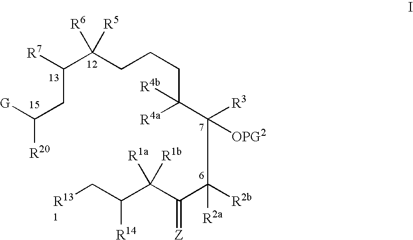
in which
R^{1a}, R^{1b} are the same or different and mean hydrogen, C₁-C₁₀-alkyl, aryl, C₇-C₂₀-aralkyl, or together mean a —(CH₂)ₘ group with m=2, 3, 4 or 5,
R^{2a}, R^{2b} are the same or different and mean hydrogen, C₁-C₁₀-alkyl, C₂-C₁₀-alkenyl, C₂-C₁₀-alkinyl, aryl, C₇-C₂₀-aralkyl or together mean a —(CH₂)ₙ group with n=2, 3, 4 or 5,
R³ means hydrogen, C₁-C₁₀-alkyl, aryl, C₇-C₂₀-aralkyl,
R^{4a}, R^{4b} are the same or different and mean hydrogen, C₁-C₁₀-alkyl, aryl, C₇-C₂₀-aralkyl or together mean a —(CH₂)ₚ group with p=2, 3, 4 or 5,
R⁵ means hydrogen, C₁-C₁₀-alkyl, aryl, C₇-C₂₀-aralkyl,
R⁶, R⁷ each mean a hydrogen atom, together an additional bond or together an oxygen atom,
G means a group X═CR⁸—, a bicyclic or tricyclic aryl radical,
R⁸ means hydrogen, halogen, C₁-C₂₀-alkyl, aryl, C₇-C₂₀-aralkyl, which all can be substituted,
X means an oxygen atom, two alkoxy groups OR²³, a C₂-C₁₀-alkylene-α,ω-dioxy group, which can be straight-chain or branched, H/OR⁹ or a grouping CR¹⁰R¹¹,
whereby
R²³ stands for a C₁-C₂₀-alkyl radical,
R⁹ stands for hydrogen or a protective group PG^{X},
R¹⁰, R¹¹ are the same or different and stand for hydrogen, a C₁-C₂₀-alkyl, aryl, or C₇-C₂₀-aralkyl radical, or R¹⁰ and R¹¹ together with the methylene carbon atom together stand for a 5- to 7-membered carbocyclic ring,
R¹³ means CH₂OR^{13a}, CH₂-Hal, CHO, CO₂R^{13b}, or COHal,
R¹⁴ means hydrogen, OR^{14a}, Hal, or OSO₂R^{14b},
R^{13a}, R^{14a} mean hydrogen, SO₂-alkyl, SO₂-aryl, SO₂-aralkyl or together a —(CH₂)ₒ group or together a CR^{15a}R^{15b} group,
R^{13b}, R^{14b} mean hydrogen, C₁-C₂₀-alkyl, aryl, C₁-C₂₀-aralkyl,
R^{15a}, R^{15b} are the same or different and mean hydrogen, C₁-C₁₀-alkyl, aryl, C₇-C₂₀-aralkyl, or together a —(CH₂)_{q} group,
o means 2 to 4,
q means 3 to 6,
R²⁰ means OPG³, NHR²⁹, or N₃,
Z means an oxygen atom or H/OR¹²,
whereby
R¹² is hydrogen or a protective group PG^{Z}
including all stereoisomers as well as mixtures thereof, and
free hydroxyl groups in R¹³ and R¹⁴ can be etherified or esterified, free carbonyl groups in Z and R¹³ can be ketalized, converted into an enol ether or reduced, and free acid groups in R¹³ und R¹⁴ can be converted into their salts with bases, characterized in that
a C1-C6 fragment (epothilone numbering system) of general formula A 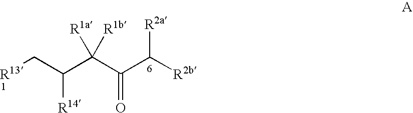
in which
R^{1a′}, R^{1b′}, R^{2a′}, R^{2b′}, R^{13′} and R^{14′} have the meanings already mentioned for R^{1a}, R^{1b}, R^{2a}, R^{2b}, R¹³ and R⁴, including all stereoisomers as well as mixtures thereof, and free hydroxyl groups in R¹³ und R¹⁴ can be etherified or esterified, free carbonyl groups in A und R¹³ can be ketalized, converted into an enol ether or reduced, and free acid groups in A can be converted into their salts with bases,
is reacted with a C7-C12 fragment (epothilone numbering system) of general formula 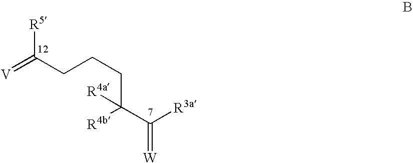
in which
R^{3a′}, R^{4a′}, R^{4b′} and R⁵ have the meanings already mentioned for R^{3a}, R⁴ and R⁵, and
V means an oxygen atom, two alkoxy groups OR¹⁷, a C₂-C₁₀-alkylene-α,ω-dioxy group, which can be straight-chain or branched, or H/OR¹⁶,
W means an oxygen atom, two alkoxy groups OR¹⁹, a C₂-C₁₀-alkylene-α,ω-dioxy group, which can be straight-chain or branched, or H/OR¹⁸,
R¹⁶, R¹⁸, independently of one another, mean hydrogen or a protective group PG¹,
R¹⁷, R¹⁹, independently of one another, mean C₁-C20-alkyl,
to form a partial fragment of general formula AB 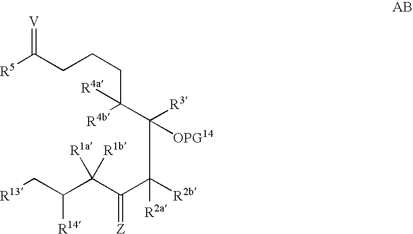
in which
R^{1a′}, R^{1b′}, R^{2a′}, R^{2b′}, R^{3′}, R^{4a′}, R^{4b′}, R⁵, R^{13′}, R^{14′}, V and Z have the already-mentioned meanings, and
PG¹⁴ represents a hydrogen atom or a protective group PG, and
this partial fragment of general formula AB is reacted with a C13-C15 fragment (epothilone numbering system) of general formula C 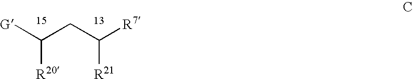
in which
G′ has the meaning already mentioned in general formula I for G, and
R^{7′} means a hydrogen atom,
R^{20′} means halogen, N₃, NHR²⁹, a hydroxy group, a protected hydroxy group O-PG³, a protected amino group NR²⁹PG³, a C₁-C₁₀-alkylsulfonyloxy group, which optionally can be perfluorinated, a benzoyloxy group that is optionally substituted by C₁-C₄-alkyl, nitro, chlorine or bromine, an NR²⁹SO₂CH₃ group, an NR²⁹C(═O)CH₃ group, or a CH₂—C(═O)—CH₃ group,
R²¹ means a hydroxy group, halogen, a protected hydroxy group OPG³, a phosphonium halide radical PPh₃⁺Hal⁻ (Ph=Phenyl; Hal=F, Cl, Br, I), a phosphonate radical P(O)(OQ)₂ (Q=C₁-C₁₀-alkyl or phenyl) or a phosphine oxide radical P(O)Ph₂ (Ph=Phenyl),
R²⁹ means hydrogen or C₁-C₆-alkyl,
to form a compound of general formula ABC (=compound of general formula I) 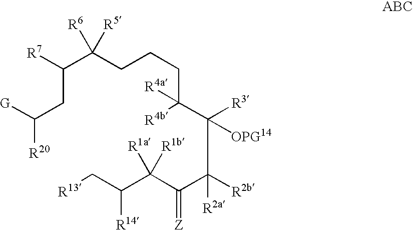
in which
R^{1a′}, R^{1b′}, R^{2a′}, R^{2b′}, R^{3′}, R^{4a′}, R^{4b′}, R^{5′}, R⁶, R⁷, R¹³, R¹⁴, G and Z have the already mentioned meanings, and
PG¹⁴ represents a hydrogen atom or a protective group PG.

2. Process according to claim 1, wherein a compound of general formula I,
in which
R^{1a}, R^{1b} are the same and mean C₁-C₆-alkyl, or together mean a —(CH2)ₘ group with m=2, 3or 4,
R^{2a}, R^{2b} are different and mean hydrogen, C₁-C₆-alkyl, C₂-C₁₀-alkenyl, C₂-C₁₀-alkinyl or C₇-C₂₀-aralkyl,
R⁵ means hydrogen, C₁-C₆-alkyl,
R⁸ means hydrogen, halogen, C₁-C₆-alkyl,
R^{15a}, R^{15b} are the same or different and mean hydrogen, C₁-C₆-alkyl, aryl, C₇-C₂₀-aralkyl, or together mean a —(CH₂)_{q} group,
q means 3 to 6,
is produced.

3. Process according to claim 1, wherein a compound of general formula I,
in which
R^{1a}, R^{1b} are the same and mean C₁-C₃-alkyl, or together mean a —(CH₂)ₘ group with m=2, 3 or 4,
R^{2a} means hydrogen,
R^{2b} means C₁-C₅-alkyl, C₂-C₆-alkenyl, or C₂-C₆-alkinyl,
R⁵ means hydrogen, or C₁-C₃-alkyl,
R⁶, R⁷ together mean an additional bond,
G means a group X═CR⁸—, or a bicyclic aryl radical,
R⁸ means hydrogen, fluorine, chlorine, or C₁-C₃-alkyl,
X means oxygen or a group CR¹⁰R¹¹,
R¹⁰ means hydrogen,
R¹¹ means aryl,
R¹³ means CH₂OR^{13a} or CO₂R^{13b},
R¹⁴ means OR^{14a},
R^{13a}, R^{14a} together mean a CR^{15a}R^{15b} group,
R^{13b} means hydrogen or C₁-C₆-alkyl,
R^{15a}, R^{15b} are the same and mean C₁-C₃-alkyl, or together mean a —(CH₂)_{q} group, or
R^{15a}, R^{15b} are different and mean hydrogen or aryl,
q means 4 or 5,
Z means oxygen,
is produced.

4. Process for the production of epothilone derivatives of general formula II 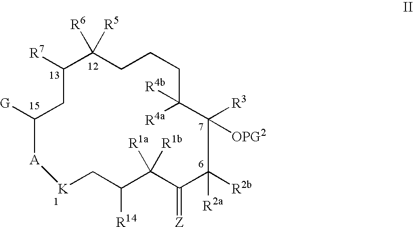
in which substituents R^{1a}, R^{1b}, R^{2a}, R^{2b}, R³, R^{4a}, R^{4b}, R⁵, R⁶, R⁷, G, OPG² and Z have the meanings that are indicated in general formula I, and
A-K means a group —O—C(═O), —OCH₂—, —CH₂C(═O)—, —NR²⁹—C(═O)—, or —NR²⁹—SO₂—, wherein an initial epothilone product of general formula I that is obtained according to claim 1 is cyclized.

5. Compounds of general formula AB 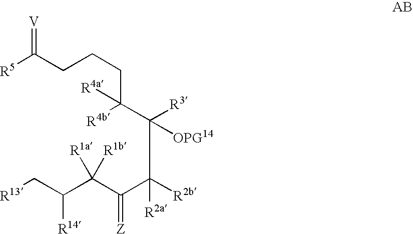
in which R^{1a′}, R^{1b′}, R^{2a′}, R^{2b′}, R^{3′}, R^{4a′}, R^{4b′}, R⁵, R^{13′}, R^{14′}, V and Z have the already mentioned meanings, and PG¹⁴ represents a hydrogen atom or a protective group PG.
